(19) United States
(12) Patent Application Publication
(10) Pub. No.: US 2022/0155314 A1
(43) Pub. Date: May 19, 2022

(54) TROPHOBLAST STEM CELL, METHODS OF PREPARATION AND USES THEREOF

(71) Applicants: Academia Sinica, Taipei (TW); MacKay Medical Foundation The Presbyterian Church In Taiwan MacKay Memorial Hospital, Taipei City (TW)

(72) Inventors: Hung-Wen Chen, Taipei (TW); Liang-Jie Wang, Taipei (TW); Chie-Pein Chen, Taipei (TW)

(21) Appl. No.: 17/530,546

(22) Filed: Nov. 19, 2021

Related U.S. Application Data
(60) Provisional application No. 63/115,652, filed on Nov. 19, 2020.

(51) Int. Cl. G01N 33/68; C12N 5/073; C12Q 1/6883

(52) U.S. Cl. CPC G01N 33/689; C12N 5/0605; C12Q 1/6883; G01N 2333/9123; G01N 2800/368; C12N 2510/00; C12Q 2600/158; C12N 2506/025

(57) ABSTRACT

An isolated pluripotent trophoblast stem (TS) cell preparation, and methods of preparing the cell preparation and a disease model for a pregnancy related disorder are provided. The cell preparation includes cells that are capable of indefinite proliferation in vitro in an undifferentiated state and capable of differentiation into cells of the trophoblast lineage in vitro or in vivo.

### BACKGROUND

### 1. Technical Field

The present disclosure relates to pluripotent trophoblast cell preparations and cell lines, methods of obtaining and maintaining the cell preparations and cell lines, and uses of the cell preparations and cell lines.

### 2. Description of Related Art

Stem cells have the capacity to divide and proliferate indefinitely in culture. Scientists aim to use these two properties of stem cells to produce seemingly limitless supplies of most human cell types from stem cells, hoping to treat diseases by cell replacement. In fact, cell therapy has the potential to treat any disease that is associated with cell dysfunction or damage including stroke, diabetes, heart attack, spinal cord injury, cancer and acquired immune deficiency syndrome (AIDS). The potential of manipulation of stem cells to repair or replace diseased or damaged tissues has generated a great deal of excitement in the scientific, medical and biotechnology investment communities. Different sources of stem cells have then been investigated and developed.

For example, embryonic stem cells (ESCs) are pluripotent cells that are capable of long-term growth, self-renewal, and can give rise to all cell types, tissues and organs. Thus, ESCs hold great promise for cell therapy as a source of diverse differentiated cell types. However, several bottlenecks of realizing such potential associated with ESCs are the risk of teratoma formation, allogenic immune rejection of ESC-derived cells by recipients, and ethical issues raised over the source for obtaining the ESCs.

The discovery of induced pluripotent stem cells (iPSC) and the direct conversion approach opened an attractive avenue that resolves these problems. The direct conversion approach and the generation of iPSCs provide an invaluable resource of cells for disease modeling, drug screening, and patient-specific cell-based therapy. However, in contrast to ESCs, the quality of iPSCs varies widely between different colonies, and a large proportion of these colonies is of low developmental potential. In addition, iPSCs are more prone to malignant transformation.

On the other hand, adult stem cells offer great opportunities for autologous cell therapy. Many different types of mesenchymal stem cells (MSCs) have been discovered and isolated from different tissues including bone marrow, peripheral blood and adipose tissue from adult and neonatal birth-associated tissues including placenta, umbilical cord and cord blood. However, heterogeneity and efficient preparation of sufficient number of MSCs remain the challenging issues in MSC-based therapies.

Therefore, there is still a need for an alternative source of adult stem cells for cell therapy. One such source is trophoblast stem cells (TSCs), which have been known as the precursors of specialized cell types of the placenta that mediate the physiological exchange between the fetus and mother during pregnancy. In the pre-implantation embryo, trophoblast cells are the first differentiated cells that can be distinguished from the pluripotent inner cell mass, and form the outermost layer of the blastocyst. MSCs have been isolated from human placentas and prepared for cell therapy. Although human TSCs have recently been derived from first-trimester placentas (TS^{CT} cells) and blastocysts (TS^{blast} cells), application of TS^{CT} and TS^{blast} cells for autologous cell therapy is unattainable (Cell Stem Cell, 22: 1-14, 2018).

A safe and stable supply of consistent trophoblast cells that can be used, for example as a source for adult stem cells, would be of great support to the development of autologous cell therapy.

### SUMMARY

This disclosure provides isolation and preparation of human trophoblast stem cells which are obtained from a term placenta. This disclosure also provides a method for obtaining human trophoblast stem cells comprising obtaining a term placenta; obtaining human placental cytotrophoblasts from the term placenta; manipulating the expression of GCM1 (glial cells missing 1) and ΔNp63α in the obtained human placental cytotrophoblasts. This disclosure also provides a composition for therapy comprising isolation and preparation of human trophoblast stem cells and a buffer solution.

In an embodiment, a pluripotent trophoblast stem cell preparation is prepared from cells obtained from a term placenta. In another embodiment, the cells obtained from the term placenta are cytotrophoblasts. In a further embodiment, the cytotrophoblasts are ITGA6 positive cytotrophoblasts.

In an embodiment, the pluripotent trophoblast stem cell preparation is capable of indefinite proliferation in vitro in an undifferentiated state. In an embodiment, the cell preparation is maintained at an undifferentiated state by manipulating the level of at least one of GCM1 and ΔNp63α or a combination thereof. In an embodiment, the level of GCM1 is suppressed. In another embodiment, the level of ΔNp63α is enhanced. In a further embodiment, the pluripotent trophoblast stem cell preparation is maintained under hypoxia condition. The cell preparation can be maintained for at least 30 passages.

In an embodiment, the pluripotent trophoblast stem cell preparation is capable of differentiation. In an embodiment, the cell preparation is capable of differentiation into cells of the trophoblast lineage. In another embodiment, the cell preparation is capable of differentiation into cells of the trophoblast lineage in vitro or in vivo. In a further embodiment, the cell preparation is capable of differentiation into multinucleated syncytiotrophoblasts (STBs) or invasive extravillous trophoblasts (EVTs).

In an embodiment, the pluripotent trophoblast stem cell preparation is further characterized by expression of TP63, TEAD4, EPCAM, HAND1 and ITGA2.

The present disclosure also provides a method for preparing the pluripotent trophoblast stem cell preparation, comprising obtaining the term placenta from a subject; isolating placental cytotrophoblasts from the term placenta; manipulating a level of at least one of glial cells missing 1 (GCM1) and A Np63c in the isolated placental cytotrophoblasts. In an embodiment, the method further comprises culturing the placental cytotrophoblasts under hypoxia condition.

The present disclosure also provides a method for establishing a disease model for a pregnancy related disorder, comprising manipulating a target gene in the pluripotent trophoblast cell preparation prepared from above and analyzing a level of a gene in the pluripotent trophoblast cell preparation. In an embodiment, manipulation of the target gene includes eliminating expression of glial cells missing 1 (GCM1) in the pluripotent trophoblast cell preparation and after analyzing a level of a gene in the pluripotent trophoblast cells and cell preparations, further comprises identifying a gene having a different level. In an embodiment, the method is for establishing a disease model for preeclampsia. In an embodiment, the gene having a different level identified in the disease model for preeclampsia are CKMT1A and CKMT1B (CKMT1). The present disclosure therefore also provides a method for diagnosing preeclampsia in a subject in need thereof, comprising obtaining a biological sample from the subject; determining a level of mitochondrial creatine kinase 1 (CKMT1) in the biological sample; comparing the level of CKMT1 with a predetermined level; determining the subject to be suffering from preeclampsia as the level of CKMT1 is lower than the predetermined level.

### BRIEF DESCRIPTION OF THE DRAWINGS

The present disclosure can be more fully understood by reading the following descriptions of the embodiments, with reference made to the accompanying drawings.

FIGS. 1A and 1B show the immunohistochemistry results of ΔNp63α and GCM1 in human placentas and FIGS. 1C to 1F show the regulation of trophoblast differentiation and stemness genes by GCM1 and ΔNp63α. FIG. 1A shows the expression of ΔNp63α and GCM1 in placentas at different gestational ages. Sections of first-trimester (gestational age 7 weeks, GA7), second-trimester (GA20), and term human placentas were immunostained with cytokeratin 7 (CK7), ΔNp63α, and GCM1 Abs, respectively. The sections were further counterstained with hematoxylin to localize the cell nuclei. FIG. 1B shows the co-expression of ΔNp63α and GCM1 in placental trophoblasts. Term human placental sections were subjected to immunostaining using ΔNp63α Ab, AP-conjugated secondary Ab, and StayGreen chromogen (Abcam, Cambridge, UK), and then GCM1 Ab, HRP-conjugated secondary Ab, and DAB chromogen (Vector Labs, Burlingame, Calif.). A higher magnification image of the boxed region is shown on the right. Arrowhead, asterisk, and arrow indicate trophoblasts expressing ΔNp63α (green), GCM1 (brown), and both, respectively.

FIGS. 1C and 1D show suppression of GCM1 target gene expression by ΔNp63α. BeWo cells were transduced with lentiviruses harboring EGFP and empty (pCDH-GFP) or ΔNp63α (pCDH-GFP-ΔNp63α-FLAG) expression cassettes, followed by flow cytometry of EGFP-positive cells. The EGFP-positive mock or ΔNp63α-expressing BeWo cells were subjected to immunoblotting and quantitative RT-PCR analyses of GCM1, hCGβ, and HTRA4 proteins and transcripts.

FIG. 1E shows downregulation of TS sternness genes by ΔNp63α knockdown. Scramble control or ΔNp63α-knockdown JEG3 cells were subjected to quantitative RT-PCR analysis of the TS cell marker genes, ELF5 and EOMES. Note that expression of GCM1 and hCGβ genes are upregulated in the ΔNp63α-knockdown cells. Means and standard deviations obtained from three independent experiments are presented.

FIG. 1F shows reciprocal expression of ΔNp63α and GCM1 in placental cells. Empty vector control (pCDH) and GCM1-HA-expressing JEG3 cells (pCDH-GCM1-HA) were treated with or without 50 μM FSK for 6 h and then harvested for immunoblotting analysis of ΔNp63α and GCM1 proteins. Arrowhead denotes a non-specific band recognized by ΔNp63α Ab.

FIGS. 2A to 2I show the physical and functional interaction between ΔNp63α and GCM1. FIG. 2A shows the co-expression of ΔNp63α and GCM1 in human placental trophoblasts. First-trimester and term placental sections were stained with ΔNp63α and GCM1 antibodies (Abs) for immunofluorescence microscopy. Boxed areas are shown at higher magnification and presented below. Arrowhead, asterisk, and arrow indicate trophoblasts expressing ΔNp63α, GCM1, and both, respectively. Nuclei were stained with DAPI. Term placental sections were stained with CK7 Ab for trophoblasts and normal mouse IgG (M-IgG) and rabbit IgG (R-IgG) for negative controls. FIG. 2B shows the physical interaction between ΔNp63α and GCM1. 293T cells were transfected with different combinations of pΔNp63α-FLAG, pOVOL1-FLAG, and pHA-GCM1 for co-immunoprecipitation analysis with HA and FLAG mAbs. FIG. 2C shows the mapping of GCM1-interacting domain in ΔNp63α (left) and ΔNp63α-interacting domain in GCM1 (right). 293T cells were transfected with pHA-GCM1 and different pΔNp63α-FLAG plasmids encoding full-length or deletion mutant ΔNp63α-FLAG proteins, followed by co-immunoprecipitation analysis with HA and FLAG mAbs. Schematic representation of functional domains in ΔNp63α is presented. DBD: DNA-binding domain; OD: oligomerization domain; SAM: sterile alpha motif; TID: transactivation inhibitory domain. In a separate experiment, 293T cells were transfected with pΔNp63α-HA and pGAL4-FLAG or different pGAL4-GCM1-FLAG plasmids encoding full-length or deletion mutant GAL4GCM1-FLAG proteins for co-immunoprecipitation analysis with HA and FLAG mAbs. Schematic representation of functional domains in GCM1 is presented. TAD: transactivation domain. FIG. 2D shows the direction interaction between GCM1 and ΔNp63α. Glutathione-conjugated agarose beads pre-bound with GST, GST-SAM or GST-OD were incubated with recombinant GCM1-FLAG protein in pull-down assays. The lower panel is Coomassie brilliant blue staining of GST fusion proteins used in pull-down assays. FIG. 2E shows the expression of GCM1 and ΔNp63α in trophoblast cell lines. Human JAR, JEG3, and BeWo trophoblast cells were subjected to immunoblotting analysis using ΔNp63α and GCM1 Abs. FIG. 2F shows the nuclear co-localization of ΔNp63α and GCM1. BeWo cells stably expressing ΔNp63α-FLAG were subjected to co-immunoprecipitation analysis with GCM1 Ab and FLAG mAb or immunofluorescence microscopy with GCM1 and ΔNp63α Abs and AlexaFluor 488-conjugated and AlexaFluor 568-conjugated secondary Abs. FIG. 2G shows that ΔNp63α suppresses GCM1 target gene expression. Mock and ΔNp63α-FLAG-expressing BeWo cells were subjected to immunoblotting (left) and quantitative RT-PCR (right) analyses of GCM1, HTRA4, and hCGβ proteins and transcripts. FIG. 2H shows that GCM1 knockdown increases ΔNp63α expression. BeWo cells stably expressing scramble or GCM1 shRNA were subjected to immunoblotting (left) and quantitative RT-PCR (right) analyses of GCM1 and ΔNp63α proteins and transcripts. FIG. 2I shows that cAMP suppresses trophoblast stemness gene expression. JEG3 cells were mock treated (DMSO or control buffer (CTRL)) or treated with 50 μM FSK or 1 mM DB-cAMP for 24 h, followed by immunoblotting and quantitative RT-PCR analyses of the indicated trophoblast differentiation and stemness proteins or transcripts. Arrowhead in FIGS. 2E, 2H, and 2I denotes a non-specific band recognized by ΔNp63α Ab.

FIGS. 3A to 3I show reciprocal regulation of GCM1 and ΔNp63α activities in trophoblast differentiation. FIG. 3A shows the regulation of trophoblast differentiation genes by ΔNp63α and GCM1. Scramble control, ΔNp63α-knockdown or GCM1-knockdown JEG3 cells were treated with or without 50 μM FSK for 24 h and then harvested for immunoblotting and quantitative RT-PCR analyses of SYN1, hCGβ, HTRA4 proteins or transcripts. Arrowhead denotes a non-specific band recognized by ΔNp63α Ab. FIG. 3B shows the enhancement of FSK-stimulated cell fusion by ΔNp63α knockdown. Scramble control and ΔNp63α knockdown JEG3 cells were treated with or without 50 μM FSK for 48 h, followed by immunofluorescence microscopy with E-cadherin Ab. Syncytial margins are marked with stippled line. Cell fusion was quantified by fusion index. FIG. 3C shows that ΔNp63α activates GATA3 gene expression. Mock and ΔNp63α-FLAG-expressing BeWo cells or scramble control and ΔNp63α-knockdown JEG3 cells were subjected to immunoblotting and quantitative RT-PCR analyses of RACK1 and GATA3 proteins or transcripts. FIG. 3D shows the suppression of HTRA4 promoter activity by ΔNp63α through GATA3. Scramble control or ΔNp63α-knockdown JEG3 cells were transfected with pHTRA4-1 Kb with or without pGATA3-FLAG for 48 h, followed by luciferase assays. FIG. 3E shows the suppression of ΔNp63α-mediated transcriptional activation by GCM1. Hep3B cells were transfected with pΔNp63α-FLAG and increasing amounts of pHA-GCM1 plus the reporter plasmid pGL3-p63bswtLuc or pGL3-p63bsmtLuc (left) or pGL3-ΔNp63αLuc (right). At 48 h post-transfection, cells were harvested for luciferase assays. FIG. 3F shows the enhanced ΔNp63α degradation by cAMP. JEG3 cells were treated with or without 50 μM FSK for 24 h in the presence or absence of MG132. Cells were harvested for immunoblotting analysis of ΔNp63α and GCM1. FIG. 3G shows the impairment of ΔNp63α oligomerization by GCM1. 293T cells were transfected with pΔNp63α-FLAG, pΔNp63α-Myc without or with pHA-GCM1 at increasing amounts for coimmunoprecipitation analysis with FLAG, HA, and Myc mAbs. FIGS. 3H and 3I show that GCM1 is required for FSK-stimulated trophoblast differentiation and ΔNp63α destabilization. GCM1-knockout (KO) JEG3 cells were generated by CRISPR/Cas9. Sequence of shRNA is underlined and the mutant sequences in GCM1 locus are highlighted with blue shaded rectangles in FIG. 3H. WT and GCM1-KO JEG3 cells were treated with or without 50 μM FSK for 24 hours and then harvested for immunoblotting and quantitative RT-PCR analyses of GCM1, ΔNp63α, hCGβ proteins and transcripts. In FIG. 3I, WT and GCM1-KO JEG3 cells were treated with 75 μM cycloheximide (CHX) and chased for the indicated periods of time. Cells were harvested for coimmunoprecipitation analysis with ΔNp63α Ab. Quantitation of band intensity was performed by densitometry analysis, and the relative ΔNp63α protein level was normalized by β-actin. Means and standard deviations obtained from three independent experiments are presented.

FIGS. 4A to 4C show reciprocal expression of GCM1 and ΔNp63α genes in TS^{CT} and TS^{blast} cells and differentiated trophoblasts. FIG. 4A shows meta-analysis of GCM1 and ΔNp63α gene expression in RNA-seq datasets (JGAS00000000107 and JGAD00000000121) of TS^{CT} and TS^{blast} cells and their derivative STBs (ST-TS cells) and EVTs (EVT-TS cells) and purified first-trimester CTBs, STBs, and EVTs (Okae et al., 2018). Heatmap representation of relative expression (Z-score) of trophoblast differentiation-associated GCM1 and GCM1 target genes (HTRA4, PGF, and CGB7) and trophoblast stemness-associated ELF5, ITGA6, and ΔNp63α genes and ΔNp63α target gene MTSS1 in the indicated cell types is shown. FIGS. 4B and 4C show meta-analysis of single-cell (sc) RNA-seq data of human first-trimester trophoblasts. scRNA-seq data were extracted from the GSE89497 dataset (Liu et al., 2018). The cell no. indicates the single-cell serial number code. Heatmap, and dot plot showing the expression levels of the selected genes in individual CTB, EVT, and STB cells are presented.

FIGS. 5A to 5C show regulation of GCM1 and ΔNp63α expression in term CTBs by EGF and small molecules (CHIR99021, A83-01, SB431542, Y27632, and VPA). FIGS. 5A and 5B show expression of GCM1 and ΔNp63α in ITGA6-positive term CTBs under TS cell culture conditions. CTBs were cultured in complete TS medium containing EGF and small molecules for 1 (FIG. 5A) or 5 (FIG. 5B) days and then subjected to immunofluorescence microscopy of GCM1 and ΔNp63α. FIG. 5C shows reciprocal regulation of GCM1 and ΔNp63α gene expression in CTBs by EGF and small molecules. CTBs in FIG. 5B were continuously cultured in complete TS medium or shifted to incomplete TS medium without EGF and small molecules for additional 7 days before immunostaining for GCM1 and ΔNp63α.

FIGS. 6A to 6I show the regulation of trophoblast stemness and differentiation by antagonism between ΔNp63α and GCM1. FIG. 6A shows the reciprocal expression of GCM1 and ΔNp63α in CTBs. ITGA6-positive term CTBs were cultured in complete TS medium for 5 days and then maintained in the same medium or incomplete medium (vehicle alone without EGF and chemical inhibitors) for an additional 7 days. Cells were subjected to immunofluorescence microscopy of ΔNp63α, GCM1, and hCGβ. FIG. 6B shows the dedifferentiation of BeWo cells. BeWo cells were incubated in complete or incomplete TS medium supplemented with the indicated growth factor or chemical inhibitor(s) for 24 h. Cells were harvested for quantitative RT-PCR analysis of the indicated stemness or differentiation genes. FIG. 6C shows the regulation of GCM1 and ΔNp63α expression by VPA. BeWo cells were treated with the indicated concentration of VPA for 24 hours and then harvested for immunoblotting and quantitative RT-PCR analyses of GCM1, ΔNp63α, and hCGβ. FIG. 6D shows the activation of Notch signaling by VPA. BeWo cells were transfected with 4XCSL-luciferase in the presence or absence of 2 mM VPA for 48 hours, followed by luciferase assays. FIG. 6E shows the interaction and co-localization of GCM1 and NotchIC. 293T cells were transfected with pHA-GCM1 and pNotch1IC-FLAG for 48 hours, followed by coimmunoprecipitation analysis and confocal microscopy using FLAG and HA mAbs and GCM1 Ab. FIG. 6F shows that NotchIC suppresses GCM1 activity. EGFP-positive mock or Notch1IC-FLAG-expressing BeWo cells were treated with or without 50 μM FSK for 24 h, followed by immunoblotting and quantitative RT-PCR analyses of GCM1, hCGβ or HTRA4. FIG. 6G shows that suppression of GCM1 expression by VPA is counteracted by MG132. BeWo cells were treated with or without 1 mM VPA in the presence or absence of 20 μM MG132 for 8 h and then subjected to immunoblotting analysis of GCM1 and Notch1IC. FIG. 6H shows the suppression of GCM1 autoregulation by NotchIC. The EGFP-positive mock or Notch1IC-FLAG-expressing BeWo cells were transfected with the GCM1 promoter reporter plasmid E1bLUCGCM1-2K for 48 h and then subjected to luciferase assays. FIG. 6I shows hypoxic regulation of GCM1 and ΔNp63α gene expression in trophoblasts. BeWo cells and ITGA6-positive term CTBs were cultured under normoxic or hypoxic conditions for 72 h and 7 days, respectively. Cells were then harvested for quantitative RT-PCR analysis of GCM1, ΔNp63α, HTRA4, hCGβ, and MKI67. Means and standard deviations obtained from three independent experiments are presented.

FIGS. 7A and 7B show that hypoxia affects TS cell proliferation and differentiation. FIG. 7A shows the bright-field images of ITGA6-positive term CTBs of the second passage (P2) cultured under normoxic and hypoxic conditions. FIG. 7B shows the expression of GCM1 and MKI67 in normoxic and hypoxic ITGA6-positive term CTBs. The P2 normoxic and hypoxic ITGA6-positive term CTBs were subjected to immunofluorescence microcopy for GCM1 and MKI67. Scale bar in FIGS. 7A and 7B, 100 μm.

FIGS. 8A to 8H show derivation of TS cells from term placentas. FIG. 8A shows the images of TS^{Term}#2 cells and their derivative EVTs and STBs. TS^{Term}#2 cells derived from ITGA6-positive term CTBs were incubated with FSK or A83-01 and NRG1 for differentiation into STBs (ST-TS^{Term}#2) or EVTs (EVT-TS^{Term}#2), respectively. Magnification of the boxed syncytium in ST-TS^{Term}#2 cells is presented to the right. FIG. 8B shows the expression of sternness markers in TS^{Term}#2 cells. TS^{Term}#2 cells were subjected to immunofluorescence microscopy for ΔNp63α, EPCAM, GATA3, TFAP2C, and MKI67. FIG. 8C shows the expression of STB markers in ST-TS^{Term}#2 cells. TS^{Term}#2 cells were induced with FSK for 5 days for differentiation into STBs and then subjected to immunofluorescence microscopy for GCM1, hCGβ, and E-cadherin (E-cad). FIG. 8D shows the differentiation of TS^{Term}#2 cells in to EVTs. TS^{Term}#2 cells were induced with A83-01 and NRG1 for 10 days for differentiation into EVTs, followed by immunofluorescence microscopy for GCM1 and HLA-G, flow cytometry analysis using HLA-G Ab or transwell invasion assay. Scale bars in FIGS. 8A to 8D, 100 μm. FIG. 8E shows the regulation of GCM1 and ΔNp63α gene expressions in TS^{Term} cells by hypoxia. TS^{Term}#2 cells and ST-TS^{Term}#2 were incubated under normoxia or hypoxia for 96 hours, followed by immunoblotting and quantitative RT-PCR analyses of GCM1, ΔNp63α, and hCGβ proteins and transcripts. FIG. 8F shows the suppression of GCM1 autoregulation by hypoxia. TS^{Term}#1 cells were transfected with pGL4-SV40 or E1bLUCGCM1-2K under normoxia or hypoxia for 48 hours and then subjected to luciferase assays. FIGS. 8G and 8H show the suppression of STB differentiation by ΔNp63α. EGFP-positive mock or ΔNp63α-expressing TS^{Term}#2 cells were treated with or without FSK for 72 hours and then subjected to quantitative RT-PCR and immunoblotting analyses of GCM1, hCGβ, SYN1, and ΔNp63α transcripts and/or proteins. Means and standard deviations obtained from three independent experiments are presented.

FIGS. 9A to 9G show the gene expression profiling of TS^{Term} cells and their derivative STBs and EVTs. FIG. 9A shows the identification of differentially expressed genes (DEGs) in TS^{Term} cells and their derivative STBs and EVTs by RNA-seq analysis. Volcano plots of DEGs between TS^{Term} cells and their derivative STBs (left) or EVTs (right) are presented. FIG. 9B shows the Pearson correlation coefficients between TS^{Term}, TS^{CT}, and TS^{blast} cell s and their derivative STBs (ST-TS^{CT}, ST-TS^{blast}, and ST-TS^{Term})ₐₙd EVTs (EVT-TS^{CT}, EVT-TS^{blast}, and EVT-TS^{Term}). FIG. 9C shows the heatmap of the expression of lineage-specific genes across TS^{Term}, TS^{CT}, and TS^{blast} cells and their derivative STBs and EVTs. Additional TS^{Term}, ST-TS^{Term}-, and EVT-TS^{Term} specific genes that match the CTB, STB, and EVT lineage-specific genes from 3D cultured human pre-gastrulation embryos are listed in the lower left part of the heatmap. FIG. 9D shows the functional annotation of DEGs in TS^{Term}, ST-TS^{Term}, and EVT-TS^{Term} cells by ConsensusPathDB. FIG. 9E shows the generation of GCM1-KO TS^{Term} cells. FIGS. 9F and 9G show that GCM1 involves in STB and EVT differentiation from TS^{Term} cells; the fusion index of GCM1-KO#6, GCM1-KO#7 and WT is shown in FIG. 9F, and percentage of HLA-G-positive cells in GCM1-KO#6, GCM1-KO#7 and WT is shown in FIG. 9G.

FIGS. 10A to 10C show in vivo differentiation potential of WT and GCM1-KO TS^{Term} cells. Engraftment of TS^{Term} (FIGS. 10A and 10B) or TS#2^{GCM1-KO#6} (FIG. 10C) cells into NOD-SCID mice are shown. NOD-SCID mice were subcutaneously injected with 5×10⁶ TS^{Term} or TS#2^{GCM1-KO#6} cells for 10 days. Sections of a TS^{Term} or TS#2^{GCM1-KO#6} cell-derived lesion were subjected to hematoxylin and eosin (H&E) staining and immunostaining of CK7, hCGβ, M-IgG, and HLA-G.

FIGS. 11A to 11I show the regulation of STB differentiation by the GCM1-CKMT1 axis. FIG. 11A shows the RNA-seq analysis of WT and GCM1-KO TS^{Term} cells. TS^{Term}#1, TS^{Term}#2, and TS#1^{GCM1-KO} cells and their derivative STBs were subjected to RNA-seq analysis. It was noted that CKMT1A and CKMT1B are barely expressed in TS^{Term} cells and upregulated in ST-TS^{Term#1} and ST-TS^{Term#2}, but not ST-TS#1^{GCM1-KO} cells. FIG. 11B shows that CKMT1 expression is upregulated in STBs. TS^{Term}#1, TS^{Term}#2, and TS^{Term}#3 cells and their derivative STBs were subjected to immunoblotting and quantitative RT-PCR analyses of GCM1, hCGβ or CKMT1 proteins and transcripts. FIG. 11C shows that GCM1 regulates CKMT1 expression. FIG. 11D shows the expression of CKMT1 in placental STBs. FIG. 11E shows the expression of CKMT1 in STB mitochondria. Scramble control and CKMT1-knockdown TS^{Term}#2 cells and their derivative STBs were stained with MitoTracker and CKMT1 Ab for immunofluorescence microscopy. Scale bars in FIGS. 11D and 11E, 100 μm. FIGS. 11F and 11G show that CKMT1 involves in STB differentiation. Scramble control and CKMT1-knockdown TS^{Term}#2 cells and their derivative STBs were subjected to immunoblotting and quantitative RT-PCR analyses of GCM1, hCGβ, LHB or CKMT1 proteins or transcripts. In a separated experiment, cells were stained with E-cadherin Ab for cell fusion analysis. Syncytial margins are marked with a stippled line. Cell fusion efficiency was measured by fusion index or the distribution of syncytia containing varying numbers of nuclei. Scale bar, 100 μm. FIG. 11H shows the regulation of STB differentiation by the creatine phosphate-CKMT1 system. Mock (ST)- or cyclocreatine-treated TS^{Term}#2 cells (ST+Cyclo-Cr) were induced into STBs for 72 h, followed by immunoblotting analysis of hCGβ protein. FIG. 11I shows that CKMT1 expression is decreased in preeclampsia. CKMT1 expression from the GSE75010 dataset of microarray analysis of 80 preeclampsia (PE) and 77 normal control women (top) Immunofluorescence microscopy of CKMT1 in normal and PE placentas are shown in the bottom, where placental sections were subjected to immunostaining with CKMT1 and CK7 Abs. Sections were then stained with a secondary Ab labeled with Alexa Fluor 568 (for CKMT1s) or Alexa Fluor 488 (for CK7). Nuclei were stained by DAPI. Insets are images of CK7 staining. White arrows point to CKMT1 expression in the STB of normal and PE placentas. Scale bar, 100 μm.

FIGS. 12A and 12B show that CKMT1 is a GCM1 target gene. ChIP-chip experiments in BeWo cells stably expressing HA-GCM1 using HA mAb (positive) or normal mouse IgG (control) revealed association of HA-GCM1 with an intron region immediately downstream of exon 1 in the CKMT1A (A) and CKMT1B (B) genes on chromosome 15q15.3. Putative GCM1-binding sites (GBSs) and their sequences are listed.

### DETAILED DESCRIPTION OF THE EMBODIMENTS

The following examples are used for illustrating the present disclosure. A person skilled in the art can easily conceive the other advantages and effects of the present disclosure. The present disclosure can also be implemented by different cases enacted, and the details of the instructions can also be based on different perspectives and applications in various modifications and changes that do not depart from the scope of the disclosure.

It is further noted that, as used in this disclosure, the singular forms “a,” “an,” and “the” include plural referents unless expressly and unequivocally limited to one referent. The term “or” is used interchangeably with the term “and/or” unless the context clearly indicates otherwise.

As used herein, “level” of a gene indicates the amount of the gene that could be found or measured in a sample or in an organism. Level or amount of a gene can be estimated through quantifying the level of amount of the products of a gene, which includes the mRNA transcribed from the gene sequence, namely the transcripts of the gene, or the protein translated therefrom. Therefore, level of a gene includes the expression level of a gene, mRNA level of a gene, or the protein level of a gene.

As used herein, the term “cell preparation” can be used interchangeably with “cell line” or “cell clone” and include a population of isolated cells whose gene levels have been artificially manipulated, for example, by culturing the cells under a formulated condition, or through genetic engineering techniques. Therefore, a cell preparation, a cell line or a cell clone of the present disclosure may be derived from or comprised of cells that have been genetically modified either in nature or by genetic engineering techniques in vivo or in vitro.

Broadly stated, the present disclosure relates to a stable pluripotent trophoblast stem (TS) cell line, cell clone or cell preparation. For example, the present disclosure may relate to a purified preparation of trophoblast stem cells which (i) are capable of indefinite proliferation in vitro in an undifferentiated state; and (ii) are capable of differentiation into cells of the trophoblast lineage in vivo. The preparation of trophoblast stem cells is also characterized by expression of genetic markers of diploid trophoblast stem cells.

A trophoblast stem cell preparation of the present disclosure may be induced to differentiate into cells of the trophoblast lineage in vitro or in vivo. The present disclosure therefore also relates to a purified trophoblast stem cell preparation of the present disclosure (e.g., cultured in vitro) induced to differentiate into cells of different lineages including the trophoblast lineage. In at least one embodiment of the present disclosure, a purified trophoblast cell preparation comprises cells of the trophoblast lineage including diploid trophoblast cells.

Cell preparations or cell lines of the present disclosure can be modified by introducing a mutation into a gene in the cells, introducing a sequence of a nucleic acid or by introducing a transgene into the cells. Insertion or deletion mutations may be introduced in a cell using standard techniques. A transgene may be introduced into cells via conventional techniques such as calcium phosphate or calcium chloride co-precipitation, DEAE-dextran-mediated transfection, lipofection, electroporation, or microinjection. Suitable methods for transforming and transfecting cells can be found in Sambrook et al. (Molecular Cloning: A Laboratory Manual, 2nd Edition, Cold Spring Harbor Laboratory press (1989)), and other laboratory textbooks. By way of example, a transgene may be introduced into cells using an appropriate expression vector including but not limited to a cosmid, a plasmid, or a modified virus (e.g., replication defective retroviruses, adenoviruses and adeno-associated viruses). Transfection is easily and efficiently obtained using standard methods including culturing the cells on a monolayer of virus-producing cells.

Provided is a method for an isolating preparation of human trophoblast stem cells which are obtained from a full term placenta. In at least one embodiment of the present disclosure, the human trophoblast stem cells are prepared from the human placental cytotrophoblasts (CTBs), which behave as stem cells capable of differentiating into multinucleated syncytiotrophoblasts (STBs) or invasive extravillous trophoblasts (EVTs). Glial cells missing 1 (GCM1) regulates STB and EVT differentiation by modulation of trophoblastic fusion, invasion, and hormone production. ΔNp63α maintains stratified epithelial stem cells and is the most abundant p63 isoform in CTBs. As provided herein, functional antagonism between GCM1 and ΔNp63α modulates trophoblast stemness and differentiation, and the modulation of GCM1 and ΔNp63α is used to maintain human trophoblast stem cells at an undifferentiated state or induced into differentiated states.

As disclosed herewith, ΔNp63α inhibits GCM1 activity through upregulation of GATA binding protein 3 (GATA3), whereas GCM1 jeopardizes ΔNp63α oligomerization, stability, and autoregulation. The combination of epidermal growth factor (EGF) with chemical inhibitors CHIR99021, A83-01, SB431542, Y27632 and valproic acid (VPA) represses GCM1 and trophoblast differentiation, but enhances ΔNp63α and trophoblast stemness. In at least one embodiment of the present disclosure, the ΔNp63α-GCM1 antagonism is manipulated with hypoxia to abolish GCM1 expression and establish human trophoblast stem cells from term placentas (TS^{Term} cells), which exhibit bipotential differentiation capacity into STBs and EVTs.

In at least one embodiment of the present disclosure, a model for studying pregnancy related disorder is provided by the trophoblast stem cells (TS^{Term} cells) prepared. In at least one embodiment, RNA-sequencing analysis of wildtype (WT) and GCM1-knockout TS^{Term} cells identifies mitochondrial creatine kinase 1 (CKMT1) as a GCM1 target for STB differentiation in terms of trophoblastic fusion and human chorionic gonadotropin β-subunit (hCGβ) synthesis. As disclosed herewith, decreased CKMT1 expression is found and related to the pregnancy disorder preeclampsia (PE). In at least one embodiment of the present disclosure, the TS^{Term} cell establishment reveals a critical role of the creatine phosphate shuttle in STB differentiation and pathogenesis of PE.

As used herein, trophectoderm (TE) is the earliest differentiated cell lineage containing a polarized layer of epithelial cells on the outer surface of blastocyst. After implantation, trophoblast stem (TS) cells in TE proliferate and differentiate into different trophoblast subtypes in a mature placenta. Human placenta is composed of villous tissues, which are classified into floating villi immersed in the maternal blood and anchoring villi attaching the placenta onto the uterus. The epithelial compartment of placental villi contains trophoblast stem or progenitor cell-like mononuclear cytotrophoblasts (CTBs), which may differentiate into a multinucleated syncytiotrophoblast (STB) layer or highly motive extravillous trophoblasts (EVTs). The STB layer mediates the exchange of nutrient, gas, and water between fetus and mother and produces hormones and growth factors such as human chorionic gonadotropin (hCG) and placental growth factor (PGF). EVTs migrate and invade the uterine decidua and interact with maternal immune cells to protect the fetus against maternal immune surveillance or remodel uterine spiral arteries to establish uteroplacental circulation.

Stem cells of stratified and columnar epithelia have identified chemical inhibitors and growth factors for creating cell culture conditions that mimic tissue niche environments for epithelial stem cells. For example, the proliferative capacity of epithelial stem cells is increased in the presence of 3T3 feeder cells plus the Rho-associated protein kinase (ROCK) inhibitor Y27632. Dual inhibition of small mothers against decapentaplegic (SMAD) signaling by blockade of the transforming growth factor beta (TGFβ) pathway with A83-01 and the bone morphogenetic protein (BMP) pathway with dorsomorphin homologue 1 (DMH-1) enhances stable propagation of human and mouse epithelial basal cell populations. Trophoblasts are of epithelial origin.

As used herein, GCM1 is a regulator of trophoblast differentiation and transactivates syncytin, HTRA4, and hCGβ genes for trophoblastic fusion, invasion, and hCG production. Cyclic adenosine monophosphate (cAMP) signaling is one pathway in the control of human trophoblast differentiation. In at least one embodiment, cAMP stimulates protein kinase A (PKA) and calcium/calmodulin-dependent protein kinase I (CaMKI) via the cAMP-PKA-CBP/DUSP23 and cAMPEpac1-CaMKI-SENP1/HDAC5 signaling cascades to phosphorylate and activate GCM1.

As used herein, the p53 family of transcription factors is composed of p53, p63, and p73. p53 is a tumor suppressor, and the gene is frequently mutated in human cancers. Neurological, pheromonal, and inflammatory defects are noted in p73-knockout mice. Alternative promoter usage and splicing generate p63 isoforms containing or lacking (ΔN) an N-terminal acidic transactivation domain (TAD) and harboring different C-terminal domains (φ, β or γ). ΔNp63α is predominantly expressed in the stem cells of stratified epithelia and is required for epidermal morphogenesis and homeostasis. In at least one embodiment, ΔNp63α is the main p63 isoform expressed in TS cell-like CTBs and is shown to suppress EVT differentiation and JEG3 cell migration.

In at least one embodiment of the present disclosure, the regulatory mechanism of human TS cell maintenance and differentiation is provided. GCM1 regulates trophoblast differentiation, and ΔNp63α maintains epithelial stem cells. In at least one embodiment, functional interaction between GCM1 and ΔNp63α determines the fate of TS cell-like CTBs. As disclosed herein, the physical interaction between GCM1 and ΔNp63α interferes with ΔNp63α oligomerization and decreases ΔNp63α stability and autoregulation. Correspondingly, cAMP stimulates trophoblast differentiation by decreasing ΔNp63α activity through GCM1. In contrast, ΔNp63α may counteract GCM1 activity through trans activation of GATA3, which interacts with and inhibits GCM1 transcriptional activity. In at least one embodiment, a combination treatment of EGF and chemical inhibitors, including CHIR99021, A83-01, SB431542, Y27632, and VPA, suppresses GCM1 to enhance ΔNp63α activity and trophoblast stemness. In at least one embodiment, VPA alone activates the Notch signaling pathway, in which the Notch intracellular domain (NotchIC) binds GCM1 and suppresses its activity. In at least one embodiment, GCM1-ΔNp63α antagonism is manipulated to completely abolish GCM1 expression by hypoxia and derive TS cells (TS^{Term}) from term placentas.

In at least one embodiment of the present disclosure, a model for studying pregnancy related disorder is provided by the trophoblast stem cells (TS^{Term} cells) prepared. RNA-sequencing analysis of wild-type (WT) and GCM1-knockout TS^{Term} cells identifies CKMT1 as a GCM1 target gene, which is a regulator in the creatine phosphate shuttle system. In at least one embodiment, suppression of CKMT1 by RNAi or cyclocreatine impedes trophoblastic fusion and hCGβ synthesis in the STB differentiation process from TS^{Term} cells. In at least one embodiment, CKMT1 expression is decreased in preeclampsia (PE) from meta-analysis of women with or without PE and in preeclamptic STBs.

Many examples have been used to illustrate the present disclosure. The examples below should not be taken as a limit to the scope of the disclosure.

### EXAMPLES

### Plasmid Constructs

Human ΔNp63α cDNA fragment with a C-terminal FLAG, HA or Myc tag was cloned into pcDNA3.1 (Invitrogen, Carlsbad, Calif.) or pCDH containing an expression cassette of puromycin resistance gene or EGFP (SBI, Mountain View, Calif.) to generate pΔNp63α-FLAG (SEQ ID NO.: 1), pΔNp63α-HA (SEQ ID NO.: 2), pΔNp63α-Myc (SEQ ID NO.: 3) or pCDH-ΔNp63α-FLAG (SEQ ID NO.: 4), respectively. Human OVOL-1 cDNA fragment with a C-terminal FLAG was cloned into pcDNA3.1 to generate pOVOL-1-FLAG (SEQ ID NO.: 5). Deletion mutant constructs of ΔNp63α harboring different functional domains were derived from pΔNp63α-FLAG. The expression plasmids pHA-GCM1, pCDH-HA-GCM1, pGATA3-FLAG, pGAL4, pGAL4-GCM1-FLAG and its deletion mutants have been described previously (Chiu, 2016 and Chang, 2005). The pCDH-Notch1IC-FLAG (SEQ ID NO.: 6) expression plasmid encoding mouse Notch1IC with a C-terminal FLAG was constructed from the mNotchIC plasmid kindly provided by Dr. R. Kopan (Washington University, St. Louis, Mo.). The pHTRA4-1 Kb and E1bLUCGCM1-2K reporter plasmids harboring human HTRA4 and GCM1 promoters have been described previously (Chiang, 2009 and Wang, 2012). Human ΔNp63α genomic fragment containing nucleotides 823 to +262 relative to the transcription start site was cloned into pGL3-basic (Promega, Wis., USA) to generate the pGL3-ΔNp63αLuc (SEQ ID NO.: 7) reporter plasmid. The pGL3-p63bswtLuc (SEQ ID NO.: 8) and pGL3-p63bsmutLuc (SEQ ID NO.: 9) reporter plasmids were constructed by cloning into pGL3-basic two copies of WT and mutant p63-binding sites derived from the p63 target gene MTSS1. Lentiviral pLKO.1-Puro short hairpin (sh) RNA expression plasmids for scramble, GCM1, ΔNp63α, and CKMT1 were obtained from the National RNAi Core Facility of Taiwan (Taipei, Taiwan). The shRNA target sequences are listed in Table 1 below.

TABLE 1
List of shRNA target sequences

| Gene | shRNA target sequence | SEQ ID NO. |
|---|---|---|
| Scramble | 5′-CCTAAGGTTAAGTCGCCCTCG-3′ | 10 |
| GCM1 | 5′-CCTCAGCAGAACTCACTAAAT-3′ | 11 |
| ΔNp63α | 5′-AGTTGCACTTATTGACCATTT-3′ | 12 |
| CKMT1A | 5′-TGAGGAGACCTATGAGGTATT-3′ | 13 |

### Cell Culture, Transfection, and Lentivirus Transduction

Cultures of 293T, BeWo, and JEG3 cells were performed as previously described (Chiu, 2016). Hep3B cells were obtained from the American Type Culture Collection (Manassas, Va.). For transient expression, cells were transfected with the indicated reporter and expression plasmids using the Lipofectamine 2000 reagent (Invitrogen). Luciferase assays were performed as previously described (Chen, 2000). For stable expression of exogenous ΔNp63α-FLAG or HA-GCM1, cells were infected with recombinant lentivirus strains harboring pCDH-ΔNp63α-FLAG or pCDH-HA-GCM1. To establish control, ΔNp63α, GCM1 or CKMT1 knockdown cells were infected with recombinant lentivirus strains harboring a scrambled, ΔNp63α, GCM1 or CKMT1 shRNA, respectively. The infected cells were subjected to antibiotic selection using 10 μg/ml of puromycin or flow cytometry, and the puromycin-resistant clones or EGFP-positive cells were pooled for studies.

### Trophoblast Stem Cells and Trophoblast Differentiation

Placental tissues were collected from healthy women undergoing elective termination of pregnancy or caesarean section. Written informed consent was obtained from each participant, and the study was approved by the Institutional Review Board of Mackay Memorial Hospital of Taiwan. To purify ITGA6-positive CTBs, villous tissues of term placenta were collected, trypsinized, and subjected to Percoll gradient centrifugation to enrich trophoblasts, which were further sorted out by flow cytometry using ITGA6 antibody (Ab) and Alexa Fluor 568-conjugated secondary Ab in a BD FACSAria IIIu sorter (BD Biosciences, San Jose, Calif.). The ITGA6-positive CTBs were seeded onto culture plates pre-coated with 5 mg/ml Col IV in complete TS cell medium (DMEM/F12 supplemented with 0.1 mM 2-mercaptoethanol, 0.2% FBS, 0.3% BSA, 0.5% penicillin-streptomycin, 1% ITS-X supplement, 50 μg/ml L-ascorbic acid, 1× EmbryoMax Nucleosides, and 50 ng/ml EGF plus chemical inhibitors (5 μM CHIR99021, 0.5 μM A83-01, 1 μM SB431542, 0.8 mM VPA, and 5 μM Y27632) modified from Okae et al. (Okae, 2018) at 37° C. under hypoxic (1% 02, 5% CO₂, and 94% N₂) conditions. Highly proliferative TS^{Term} cells were established after three or four passages and were used for analysis of GCM1 and ΔNp63α expression after passage 10.

Induction of STBs or EVTs from TS^{Term} cells was performed by incubation of TS^{Term} cells with ST or EVT medium as described by Okae et al. under normoxic conditions.

To study the effect of EGF and chemical inhibitors on trophoblast differentiation, BeWo cells were cultured in the complete TS medium or in the incomplete TS medium with EGF alone or the indicated chemical inhibitor(s) for 24 h. Cells were then harvested for quantitative RT-PCR or immunoblotting analysis of expression of ΔNp63α, eomesodermin (EOMES), E74-like factor 5 (ELF5) or GCM1 and its target genes.

GCM1-knockout JEG3 or TS^{Term} cells were generated by the CRISPR/Cas9 system. In brief, JEG3 or TS^{Term} cells were infected with lentiviruses harboring the pAll-Cas9.Ppuro vector (provided by the National RNAi Core Facility of Taiwan) with a gRNA sequence (5′-CAGGAAGGCGTCCAATTGCC-3′ (SEQ ID NO. 48)) targeting exon 6 of the human GCM1 gene. After puromycin selection, the surviving cells were seeded individually into 96 wells, and genomic DNA of each single colony was extracted for PCR amplification and sequencing of the gRNA targeting site.

### Immunofluorescence Microscopy and Cell Fusion Assay

First-trimester and full-term human placental tissues were fixed with formalin and embedded in paraffin wax and sectioned as described previously (Cheong, 2016). The sections were deparaffinized, rehydrated, and incubated with IgG, rabbit antiGCM1 Ab or ΔNp63α mAb (Abcam, Cambridge, UK) and then MaxFluor 488 secondary Ab for mouse IgG and MaxFluor 550 secondary Ab for rabbit IgG according to the manufacturer's instructions (MaxVision Biosciences, Kenmore, Wash.). Nuclei were stained with 4′,6-diamidino-2-phenylindole (DAPI) Immunofluorescence was examined under an Olympus laser scanning confocal microscope (FV3000) (Shinjuku, Tokyo, Japan). Images were prepared for presentation using Adobe Photoshop v7.0.

For co-localization of GCM1 and ΔNp63α, BeWo cells stably expressing ΔNp63α-FLAG were fixed in 4% paraformaldehyde and stained with GCM1 Ab and FLAG mAb, followed by incubation of AlexaFluor 488- or AlexaFluor 568-conjugated secondary Ab.

For cell fusion analysis, scramble or ΔNp63α shRNA-expressing JEG3 cells treated with or without 50 μg/ml forskolin (FSK) for 48 h, followed by immunofluorescence staining with E-cadherin Ab (BD Biosciences) and AlexaFluor 568-conjugated secondary Ab Images were captured by the aforementioned confocal microscope. Three microscopic fields per sample were randomly selected for examination in each of three independent experiments. Quantification of cell fusion was calculated as a fusion index of (N−S)/T, where N is the number of nuclei in the syncytia, S is the number of syncytia, and T is the total number of nuclei counted. In addition, cell fusion efficiency was measured by distribution of syncytia of different sizes (containing varying numbers of nuclei) as a ratio of the total number of nuclei per syncytium size over the total number of syncytial nuclei counted.

### Co-Immunoprecipitation and Pull-Down Assays

To study the interaction between GCM1 and ΔNp63α or OVOL1, 293T cells were transfected with pHA-GCM1 and pΔNp63α-FLAG or pOVOL1-FLAG. At 48 h post-transfection, cells were harvested in lysis buffer containing 50 mM Tris-HCl (pH 8.0), 150 mM NaCl, 2 mM EDTA, 10% glycerol, 0.5% NP-40, 1 mM DTT, 5 mM NaF, 1 mM Na₃VO₄, 1 mM PMSF, and a protease inhibitor cocktail (Sigma-Aldrich), followed by consecutive immunoprecipitation and immunoblotting with FLAG and HA (Sigma-Aldrich) mAbs. To map the ΔNp63α domain that interacts with GCM1, 293T cells were transfected with pHA-GCM1 and pΔNp63α-FLAG or its derivatives harboring full-length ΔNp63α or deletion mutants of ΔNp63α. The proteins were immunoprecipitated with FLAG mAb, followed by immunoblotting with HA mAb. Likewise, to map the ΔNp63α-binding domain in GCM1, 293T cells were transfected with pΔNp63α-HA and pGal4-FLAG or pGal4-GCM1-FLAG harboring full-length GCM1 or deletion mutants of GCM1. The proteins were immunoprecipitated with HA mAb, followed by immunoblotting with FLAG mAb. Pull-down assays were performed to confirm the interaction between GCM1 and ΔNp63α, and recombinant GCM1-FLAG was first immunopurified with FLAG mAb-conjugated agarose beads (Sigma-Aldrich) from 293T cells transfected with pGCM1-FLAG.

Recombinant GCM1-FLAG was then incubated with bacterially expressed GSTΔNp63α-SAM or GST-ΔNp63α-OD, which is a glutathione S-transferase (GST) fusion protein of ΔNp63α sterile alpha motif (SAM) or oligomerization domain (OD), pre-bound to glutathione-conjugated agarose beads (GE Healthcare Biosciences, Pittsburgh, Pa.) in lysis buffer. After washing, the proteins that were pulled down were analyzed by immunoblotting with FLAG mAb.

### Quantitative RT-PCR

BeWo cells stably expressing ΔNp63α-FLAG or GCM1 shRNA were harvested for RNA isolation using the High Pure RNA Isolation Kit (Roche, Basel, Switzerland). Likewise, JEG3 cells or JEG3 cells stably expressing GCM1 or ΔNp63α shRNA were mock treated or with 50 μg/ml FSK or 1 mM DB-cAMP for 24 h and then harvested for RNA isolation. The isolated RNA was transcribed into cDNA using SuperScript III reagents (Invitrogen) with an oligo-(dT)₂₀ primer. Quantification of the transcript levels of indicated genes was performed in the LightCycler system (Roche) using a commercial SYBR Green reaction reagent (Qiagen) and specific primer sets. The sequences of the primer sets were listed in Table 2 below.

TABLE 2
List of primer set sequences

| | | SEQ ID |
|---|---|---|
| Primer | Sequence | NO. |
| ΔNp63α-F | 5′-AGGAAGAGACAGGAAGGC-3′ | 14 |
| ΔNp63α-R | 5′-TGTGTGCTGAGGAAGGT-3′ | 15 |
| ELF5-F | 5′-GCTGCGACCAGTACAAGTTG-3′ | 16 |
| ELF5-R | 5′-CTGCCTCGACGAACTCCTC-3′ | 17 |
| EOMES-F | 5′-CCTAATACTGGTTCCCACT-3′ | 18 |
| EOMES-R | 5′-CGCCATCCTCTGTAACTTC-3′ | 19 |
| TEAD4-F | 5′-GACAGAGTATGCTCGCTAT-3′ | 20 |
| TEAD4-R | 5′-CTGGCTGACACCTCAAAG-3′ | 21 |
| AXIN2-F | 5′-GTCTCCAAGCAGCTGAAGCC-3′ | 22 |
| AXIN2-R | 5′-CCTCCATCACCGACTGGATC-3′ | 23 |
| GCM1-F | 5′-AACTCCATCATGAAGTGTGACG-3′ | 24 |
| GCM1-R | 5′-GATCCACATCTGCTGGAAGG-3′ | 25 |
| HTRA4-F | 5′-TTCTGTGAGCGAGACCC-3′ | 26 |
| HTRA4-R | 5′-GGAGATTCCATCAGTCACCC-3′ | 27 |
| hCGβ-F | 5′-CTGAGTCTCTGAGGTCACTT-3′ | 28 |
| hCGβ-R | 5′-TGATAGGATGCTGGGGT-3′ | 29 |
| Syncytin-1-F | 5′-GAAGGCCCTTCATAACCAATGA-3′ | 30 |
| Syncytin-l-R | 5′-GATATTTGGCTAAGGAGGTGATGTC-3′ | 31 |
| PGF-F | 5′-TCAGAGGTGGAAGTGGTACCCT-3′ | 32 |
| PGF-R | 5′-GCAGAGGCCGGCATTC-3′ | 33 |
| WNT10B-F | 5′-TTCTGTGAGCGAGACCC-3′ | 34 |
| WNT10B-R | 5′-CATCACACAGCACATAGC-3′ | 35 |
| GATA3-F | 5′-GTCAGCACCAAACAGCG-3′ | 36 |
| GATA3-R | 5′-GGAGATTCCATCAGTCACCC-3′ | 37 |
| HLA-G-F | 5′-CGCACAGACTGACAGAAT-3′ | 38 |
| HLA-G-R | 5′-AGGTAATCCTTGCCATCGTA-3′ | 39 |
| ITGA1-F | 5′-CCTGTTCTTGATGATTCTCTACC-3′ | 40 |
| ITGA1-R | 5′-TTGACTGTGAGGCTAACG-3′ | 41 |
| FLT4-F | 5′-ACAACTGGGTGTCCTTTC-3′ | 42 |
| FLT4-R | 5′-TCTGCTCAAACTCCTCCG-3′ | 43 |
| CKMT1-F | 5′-AAAGATAGCCGCTTCCC-3′ | 44 |
| CKMT1-R | 5′-GCCGTTCACAATCAATCAAATAGTT | 45 |
| | TA-3′ | |
| UBC-F | 5′-GCTGGAAGATGGACGCA-3′ | 46 |
| UBC-R | 5′-ATTCTCAATGGTGTCACTCG-3′ | 47 |

### ΔNp63α Stability and Oligomerization

To study the effect of GCM1 on ΔNp63α stability, JEG3 cells were treated with FSK alone or plus MG132 for 24 h and then subjected to immunoblotting analysis with ΔNp63α and GCM1 Abs. In a separate experiment, 293T cells were transfected with pΔNp63α-Myc and increasing amounts of pHA-GCM1. At 24 hours post-transfection, cells were treated with or without MG132 for additional 24 h before being harvested for immunoblotting analysis using HA and Myc mAbs and ΔNp63α Ab. The half-life of ΔNp63α was compared in WT and GCM1-KO JEG3 cells in the presence of cycloheximide for different periods of time. Cells were harvested for coimmunoprecipitation analysis with ΔNp63α Ab. Densitometric analysis of immunoblot band intensities was performed using ImageJ software.

To study the effect of GCM1 on ΔNp63α oligomerization, 293T cells were transfected with pΔNp63α-Myc and pΔNp63α-FLAG plus or minus increasing amounts of pHA-GCM1. At 48 hours post-transfection, cells were harvested for co-immunoprecipitation analysis with HA, FLAG, and Myc mAbs.

### RNA Sequencing

WT and GCM1-KO TS^{Term} cells and their derivative STBs and EVTs were harvested for RNA purification using the RNeasy Mini Kit and RNase-free DNase (Qiagen, Hilden, Germany) To assess the RNA integrity, RNA integrity number (RIN) was created using RNA 6000 Nano and 2100 Bioanalyzer System (Agilent Technologies, Santa Clara, Calif.). Each sample had a RIN (RNA integrity number) value above 7. RNA-seq libraries were prepared using Universal RNA-Seq with NuQuant library preparation kit (Tecan Trading AG, Switzerland) according to the manufacturer's instructions. The libraries were sequenced on the NovaSeq 6000 platform (Illumina) to produce 45 to 49 million 2×150 bp paired-end reads per sample. RNA-seq reads were trimmed using CLC Genomics Workbench v10 to a minimum quality score of 0.01 (equivalent to Phred score of 20), and adaptors were also removed. The trimmed reads were aligned to the reference genome Homo sapiens GRChg38 using CLC Genomics Workbench v10. Gene expression was measured by FPKM (fragments per kilobase of gene/transcript model per million mapped fragments) by calculated fragments with Subread package (featureCounts, v1.6.5). Differentially expressed genes were identified using DESeq. The fold change ≥3 and P-value ≤0.05 were selected to identify differentially expressed genes.

### Statistical Analysis

Differences were assessed by the Student's t-test. A P-value of <0.05 was considered statistically significant (* P<0.05; ** P<0.01).

### Example 1: Expression of GCM1 and ΔNp63α in Placenta

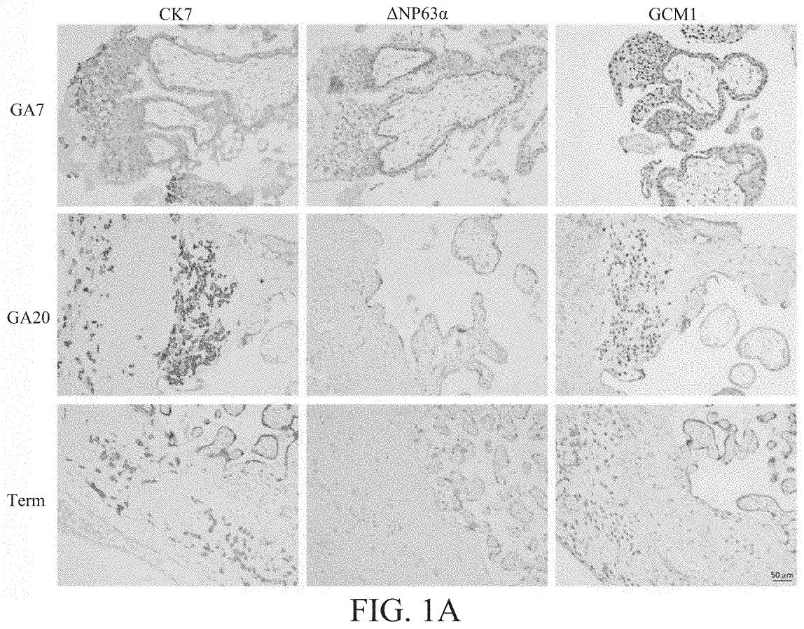

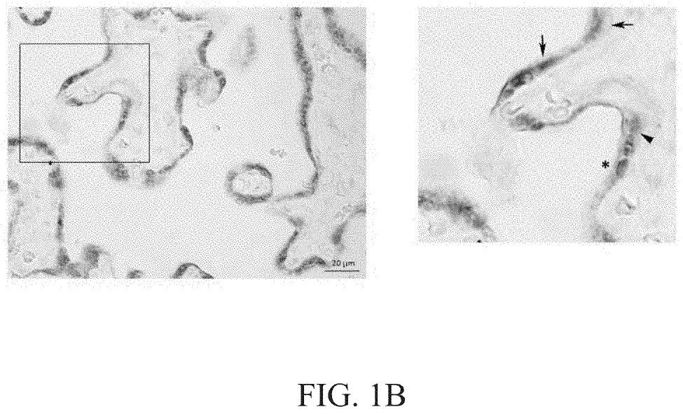

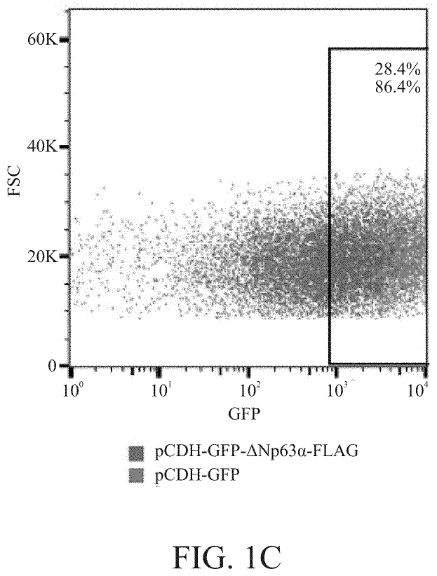

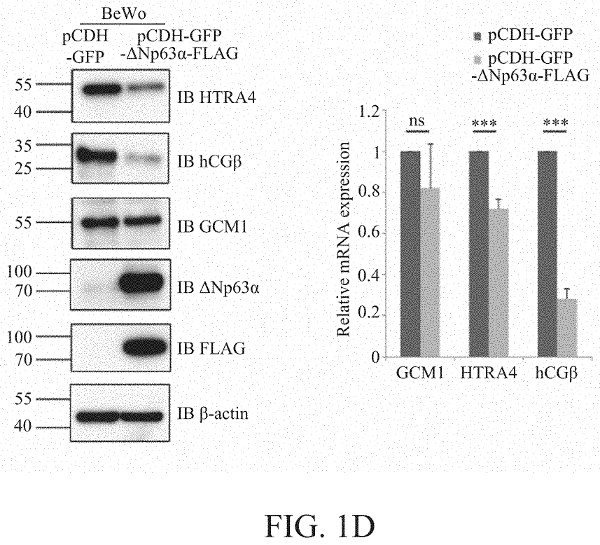

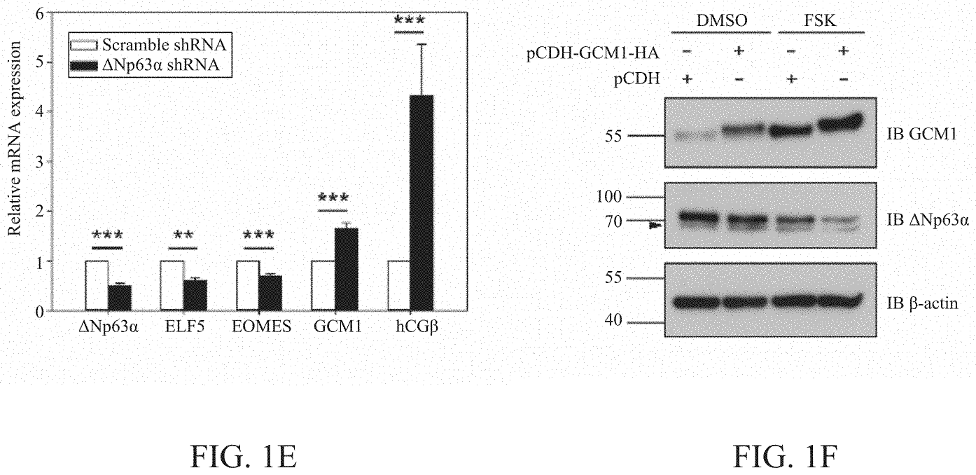

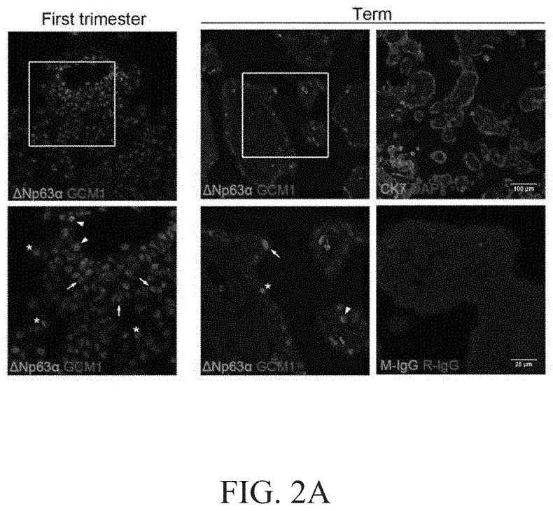

The expression patterns of GCM1 and ΔNp63α in human placentas at different gestational stages were investigated by immunohistochemistry (IHC). GCM1 expression was detected in the EVTs and STBs of different gestational stages, whereas ΔNp63α was mainly expressed in first-trimester CTBs as well as second-trimester and term CTBs and STBs as shown in FIG. 1A.

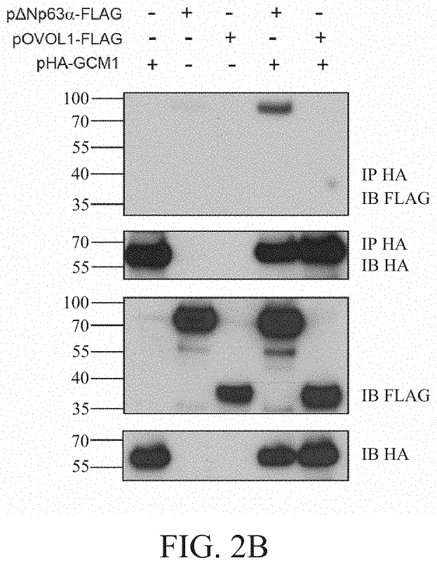

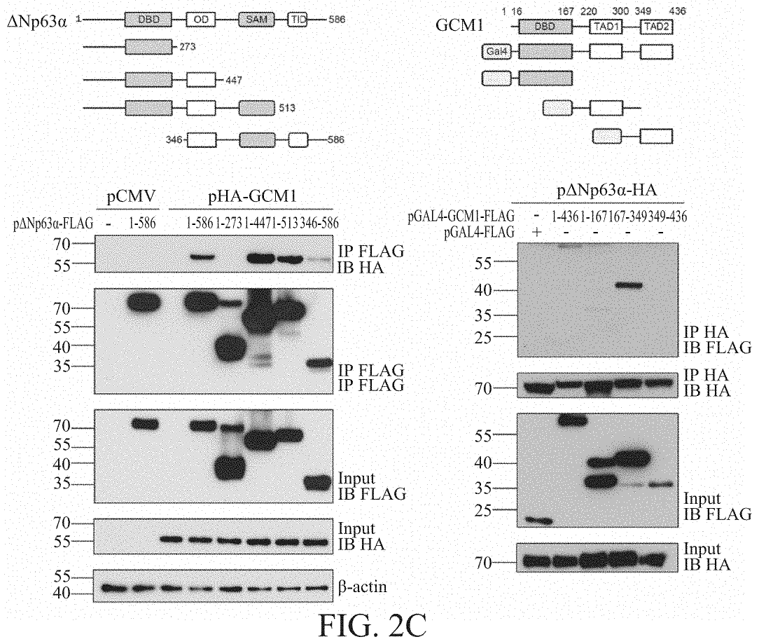

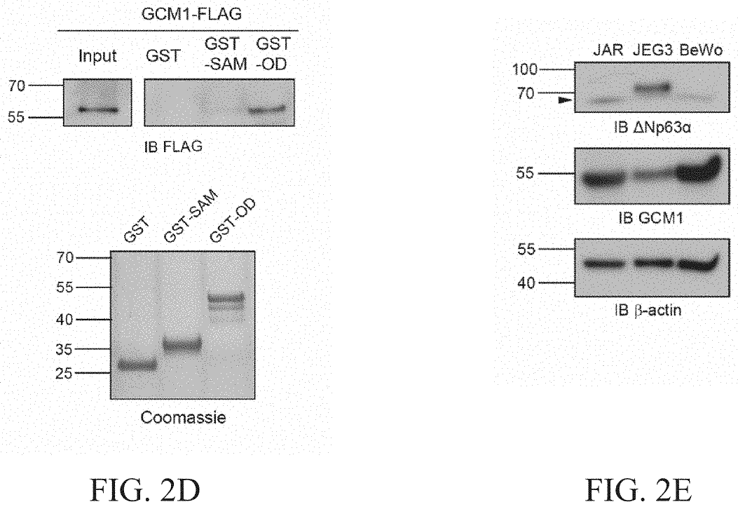

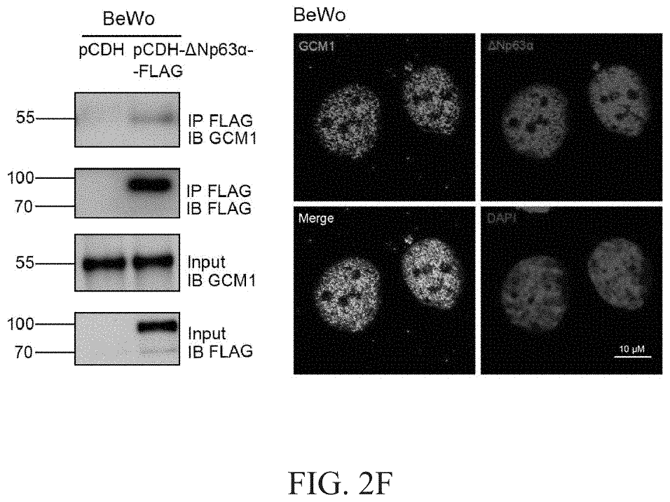

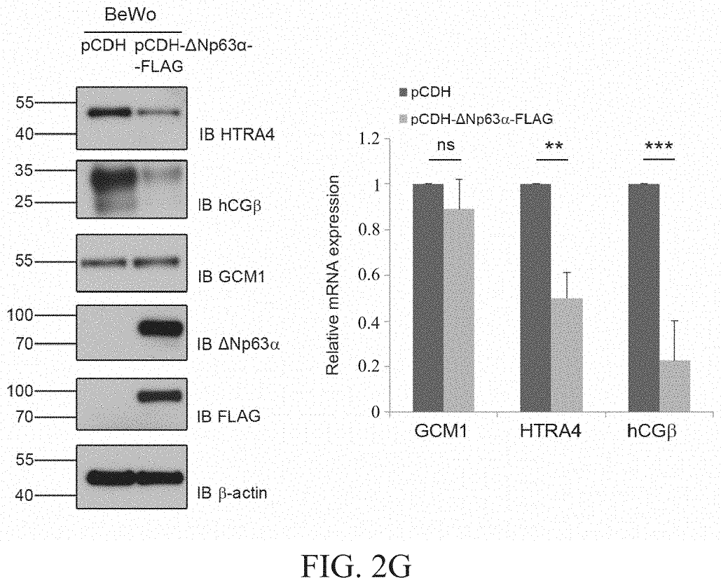

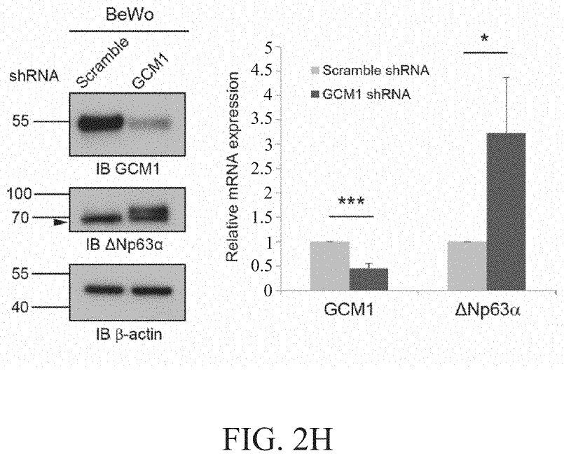

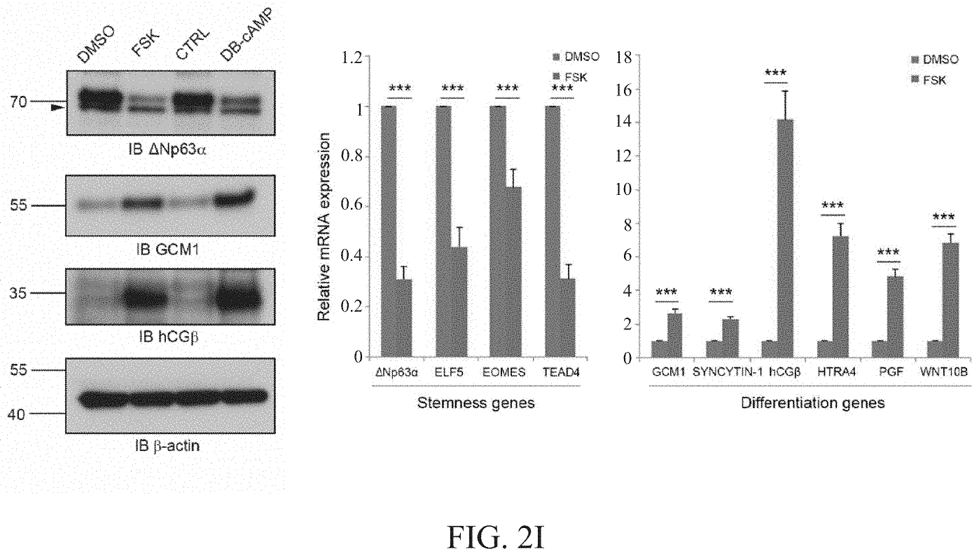

In addition, co-expression of GCM1 and ΔNp63α was observed in tested first-trimester CTBs of the proximal cell column and term STBs by immunofluorescence microscopy, as shown in FIG. 2A. To rule out methodological bias, chromogenic IHC double staining also revealed co-expression of GCM1 and ΔNp63α in term STBs, as shown in FIG. 1B.

### Example 2: Physical and Functional Interaction Between GCM1 and ΔNp63α

Interaction between GCM1 and ΔNp63α was evaluated by coimmunoprecipitation analysis in 293T cells transfected with pHA-GCM1 and pΔNp63α-FLAG or pOVOL1-FLAG, which encodes a zinc finger-containing transcription factor regulating trophoblast fusion. As shown in FIG. 2B, interaction was detected between GCM1 and ΔNp63α, but not OVOL1.

The GCM1-interacting domain in ΔNp63α was further mapped to the region between amino acids 274 and 447 in the ΔNp63α polypeptide. It corresponds to the oligomerization domain (OD), as shown in the left panel of FIG. 2C. Likewise, the ΔNp63α-interacting domain in GCM1 was mapped to the region between amino acids 167 and 349 in the GCM1 polypeptide, which harbors the transactivation domain 1 as shown in the right panel of FIG. 2C.

By GST pull-down assays using recombinant GCM1-FLAG and GST, GST-SAM or GST-OD, physical interaction was detected between GCM1-FLAG and GST-OD, but neither GST nor GST-SAM, as depicted in FIG. 2D.

The GCM1 and ΔNp63α protein levels in human trophoblast cell lines were surveyed. While JAR and BeWo cells express higher levels of GCM1 and barely express ΔNp63α, JEG3 cells express higher levels of ΔNp63α and lower levels of GCM1, suggesting an inverse relationship between ΔNp63α and GCM1 expression in trophoblasts, as shown in FIG. 2E. FIG. 2F revealed interaction and nuclear co-localization of endogenous GCM1 and ΔNp63α-FLAG through stable expression of ΔNp63α-FLAG in BeWo cells by lentiviral transduction. In the meantime, the protein and transcript levels of the GCM1 target genes HTRA4 and hCGβ were decreased in the ΔNp63α-FLAG-expressing BeWo cells, as shown in FIG. 2G. Similar observations were made in the sorted EGFP-positive BeWo cells co-expressing ΔNp63α-FLAG as shown in FIGS. 1C and 1D.

In addition, knocking down GCM1 elevates ΔNp63α transcript and protein levels in BeWo cells, suggesting a reciprocal regulation of GCM1 and ΔNp63α activity in BeWo cells, as shown in FIG. 2H.

Regarding differentiation status, JEG3 cells are likely similar to the trophoblasts co-expressing ΔNp63α and GCM1 in placenta, as shown in FIG. 2A. The cAMP stimulant FSK or dibutyryl-cAMP (DB-cAMP) is known to drive STB differentiation. Stimulation of JEG3 cells by either reagent resulted in increased expression of GCM1 and its target genes syncytin-1 (SYN1), hCGβ, HTRA4, PGF, and WNT10B with a concomitant decreased expression of ΔNp63α and trophoblast sternness genes ELF5, EOMES, and TEAD4, as shown in FIG. 2I. Correspondingly, ΔNp63α knockdown in JEG3 cells suppressed ELF5 and EOMES expression and enhanced GCM1 and hCGβ expression, as shown in FIG. 1E, whereas overexpression of GCM1-HA in JEG3 cells downregulates ΔNp63α expression, which was further enhanced by FSK (FIG. 1F).

### Example 3: Downregulation of GCM1 Activity and Trophoblast Differentiation by ΔNp63α

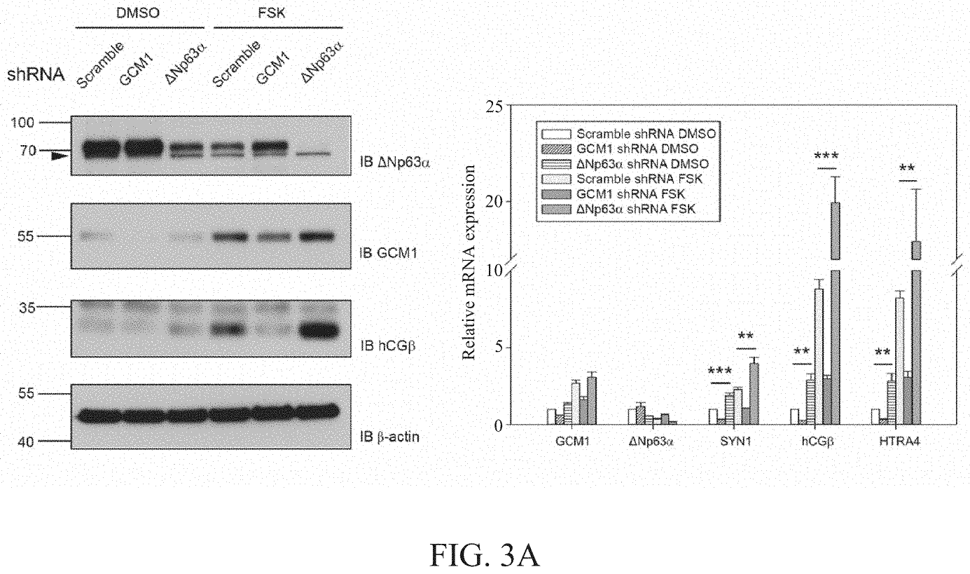

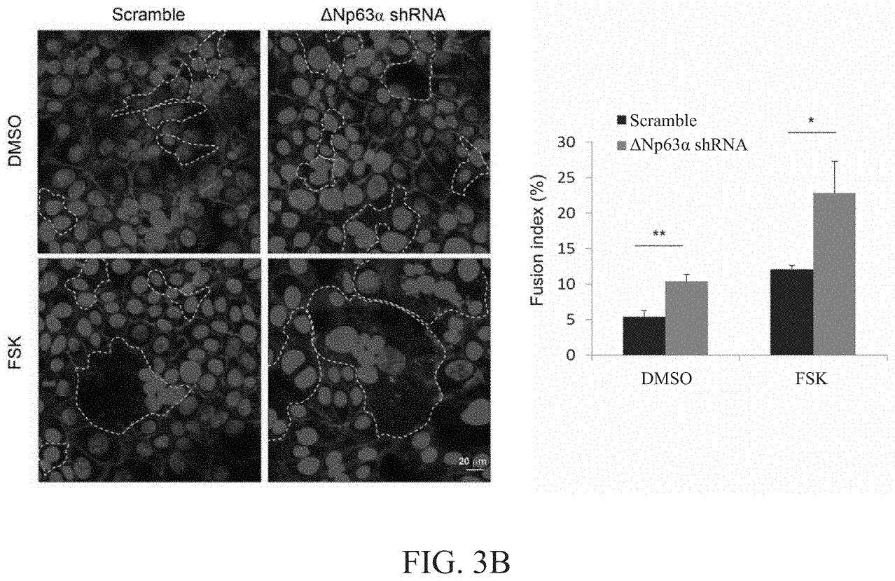

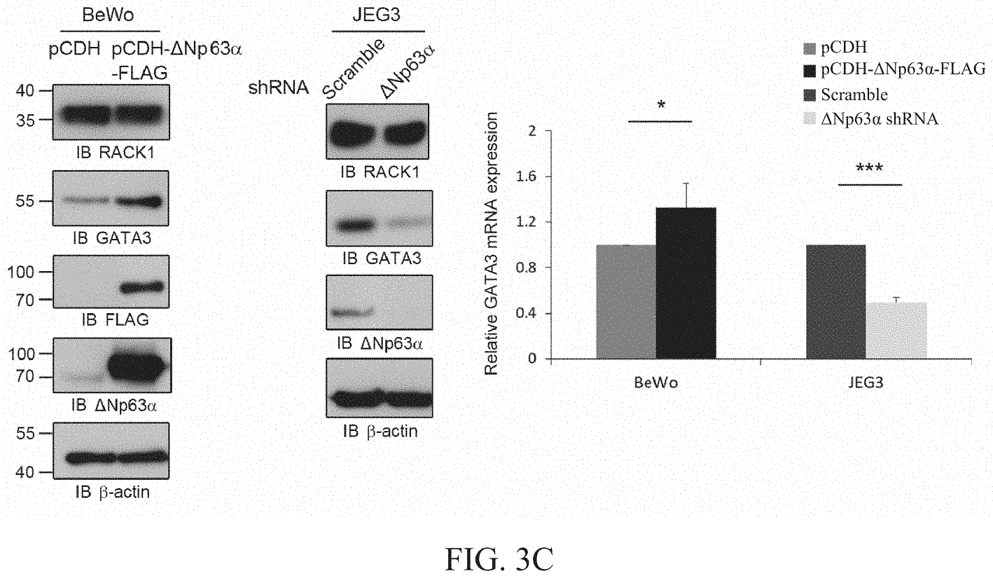

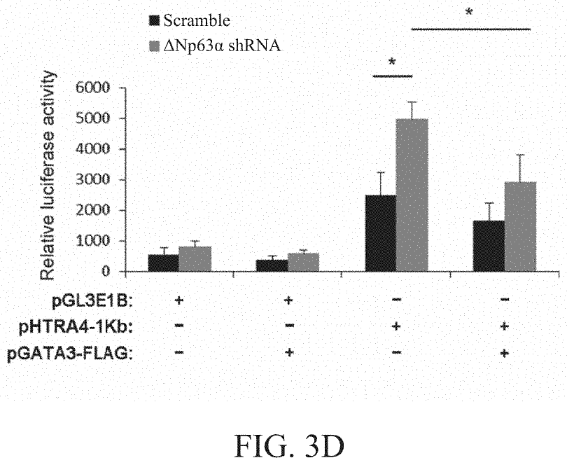

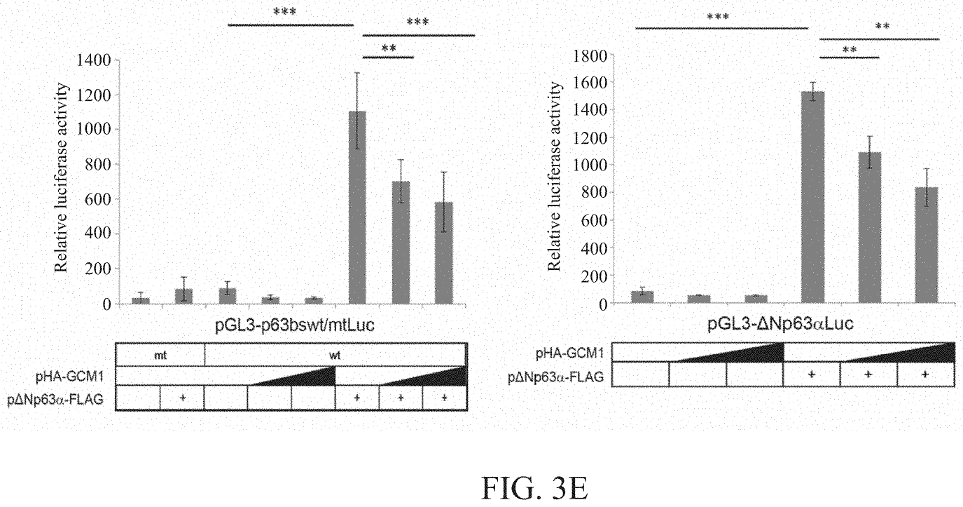

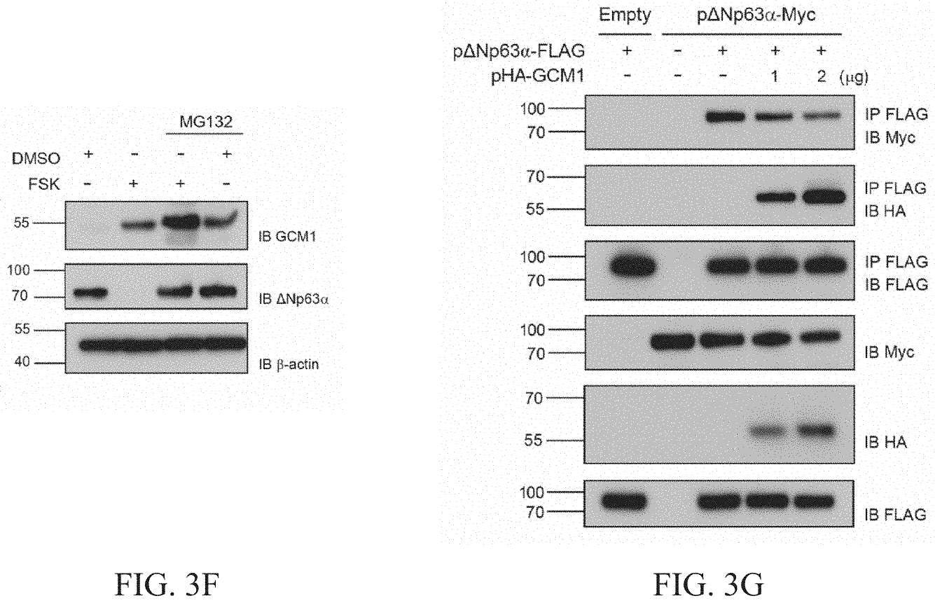

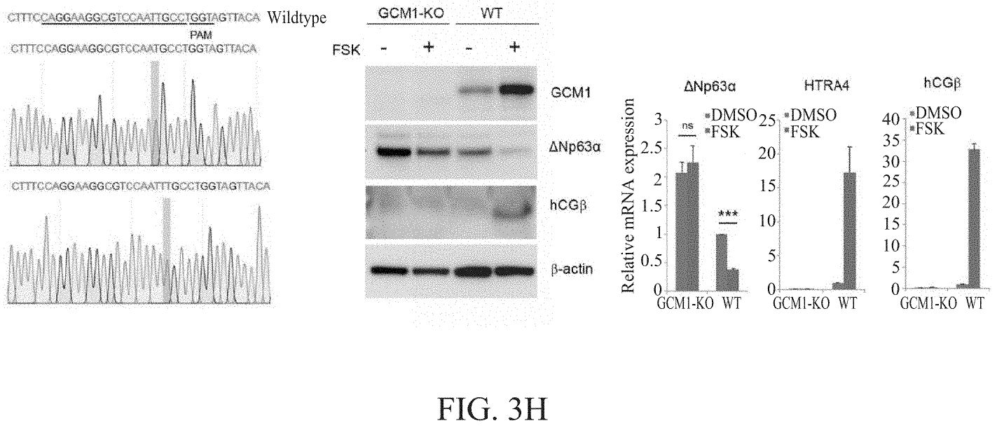

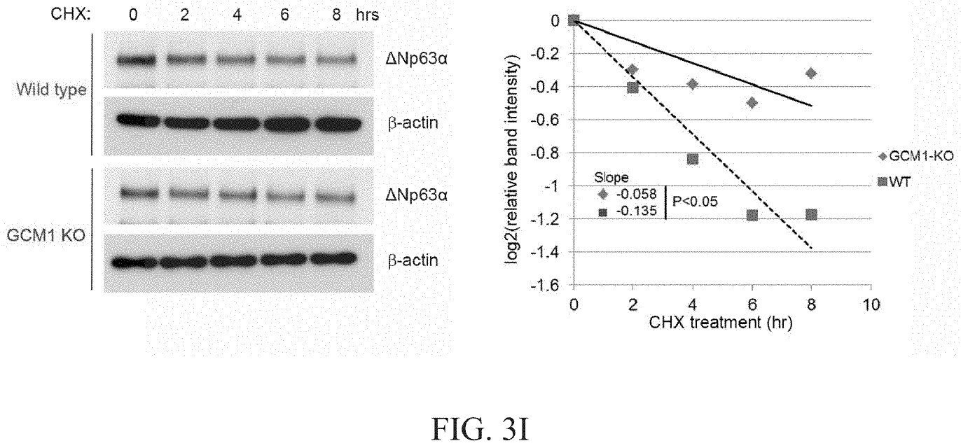

GCM1 and ΔNp63α is shown to reciprocally regulate trophoblast differentiation. The effects of GCM1 or ΔNp63α knockdown on the differentiation of JEG3 cells in response to FSK were investigated. As shown in FIG. 3A, suppression of ΔNp63α expression by FSK was counteracted by GCM1 knockdown; stimulation of SYN1, hCGβ, and HTRA4 expression by FSK was enhanced by ΔNp63α knockdown. Correspondingly, fusion of JEG3 cells stimulated by FSK was enhanced by ΔNp63α knockdown as shown in FIG. 3B. Together with the ΔNp63α overexpression study in BeWo cells shown in FIG. 2F, these results suggested that ΔNp63α downregulates GCM1 and its target genes to suppress trophoblast differentiation.

However, mammalian two-hybrid assays indicated that neither the DNA-binding activity nor the transcriptional activity of GCM1 was affected by ΔNp63α. Therefore, the interaction between ΔNp63α and GCM1 is unlikely to directly suppress GCM1 activity.

GATA3 is a ΔNp63α target gene and interacts with GCM1 to inhibit its transcriptional activity. Indeed, the transcript and protein levels of GATA3 were elevated in BeWo cells by ΔNp63α-FLAG overexpression and decreased in JEG3 cells by ΔNp63α knockdown, as shown in FIG. 3C. As a control, expression of the RACK1 scaffold protein, which interacts with and upregulates GCM1 stability, was not affected by ΔNp63α-FLAG overexpression or knockdown, as shown in FIG. 3C.

The transcriptional activity of GCM1 on the HTRA4 reporter plasmid pHTRA4-1 kb was assayed in scramble control and ΔNp63α knockdown JEG3 cells. As shown in FIG. 3D, luciferase activities directed by pHTRA41 kb were increased by ΔNp63α knockdown, which was counteracted in the presence of GATA3-FLAG. These results suggested that ΔNp63α may indirectly inhibit GCM1 activity through GATA3.

### Example 4: Downregulation of ΔNp63α Activity by GCM1

GCM1 is also shown to regulates ΔNp63α activity. A ΔNp63α-specific reporter construct pGL3-p63bswtLuc was co-transfected with pΔNp63α-FLAG and increasing amounts of pHA-GCM1 into p53-deficient Hep3B cells. As expected, ΔNp63α-FLAG did not affect luciferase activity directed by pGL3-p63bsmtLuc, which harbors mutant p63-binding sites. It was found that ΔNp63α-FLAG upregulated the luciferase activity directed by pGL3-p63bswtLuc, which was suppressed by HA-GCM1 in a dose-dependent manner, as shown in the left panel of FIG. 3E. Because of ΔNp63α autoregulation, transactivation of the ΔNp63α promoter reporter construct pGL3-ΔNp63 Luc by ΔNp63α-FLAG is also suppressed by HA-GCM1 in a dose-dependent manner as shown in the right panel of FIG. 3E. Therefore, GCM1 interacts with ΔNp63α to suppress its transcriptional activity.

Because FSK suppresses ΔNp63α expression through GCM1, as shown in FIG. 3A, further studies were carried out to elucidate how GCM1 downregulates ΔNp63α activity. Indeed, the suppressive effect of FSK on ΔNp63α□ expression was counteracted by the proteasome inhibitor MG132 in JEG3 cells, suggesting that FSK facilitates ΔNp63α degradation, as shown in FIG. 3F. However, ubiquitination of ΔNp63α was not affected by GCM1 or FSK. Oligomerization is essential for the biological functions of p53 family members in regulation of cell cycle and development. GCM1 interferes with the intermolecular interaction between ΔNp63α because the interaction between ΔNp63α-FLAG and ΔNp63α-Myc is decreased by increasing amounts of HA-GCM1 in transient expression experiments, as shown in FIG. 3G.

Further, GCM1-knockout (KO) JEG3 cells were generated by the CRISPR/Cas9 system. As expected, stimulation of hCGβ or HTRA4 expression by FSK was blunted in the GCM1-KO JEG3 cells, as shown in FIG. 3H. Compared with WT JEG3 cells, the ΔNp63α protein and transcript levels were elevated in the GCM1-KO JEG3 cells, which were not significantly affected by FSK, as shown in FIG. 3H. By cycloheximide chase assay, it was further demonstrated that ΔNp63α stability is increased in the GCM1-KO JEG3 cells, as shown in FIG. 3I. These results suggested that GCM1 inhibits ΔNp63α activity by blocking ΔNp63α oligomerization, which may result in ubiquitin-independent proteasome degradation of ΔNp63α.

### Example 5: Antagonism Between GCM1 and ΔNp63α Controls Trophoblast Stemness

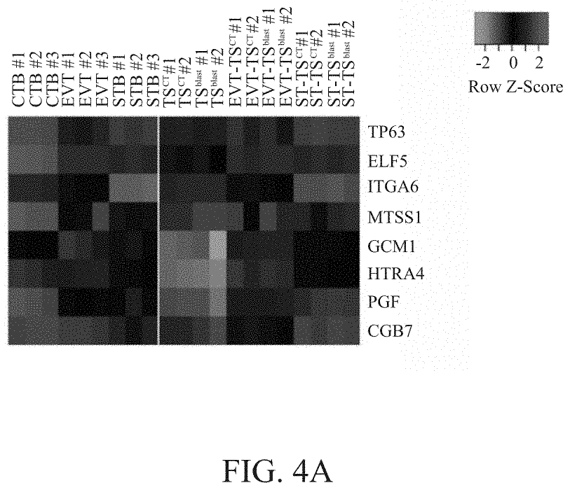

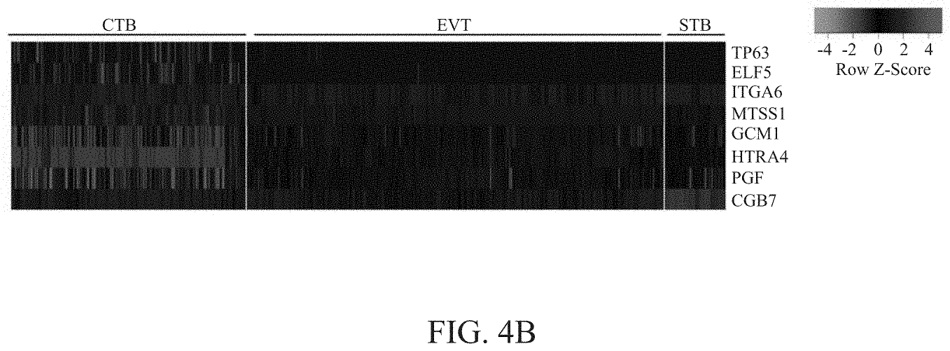

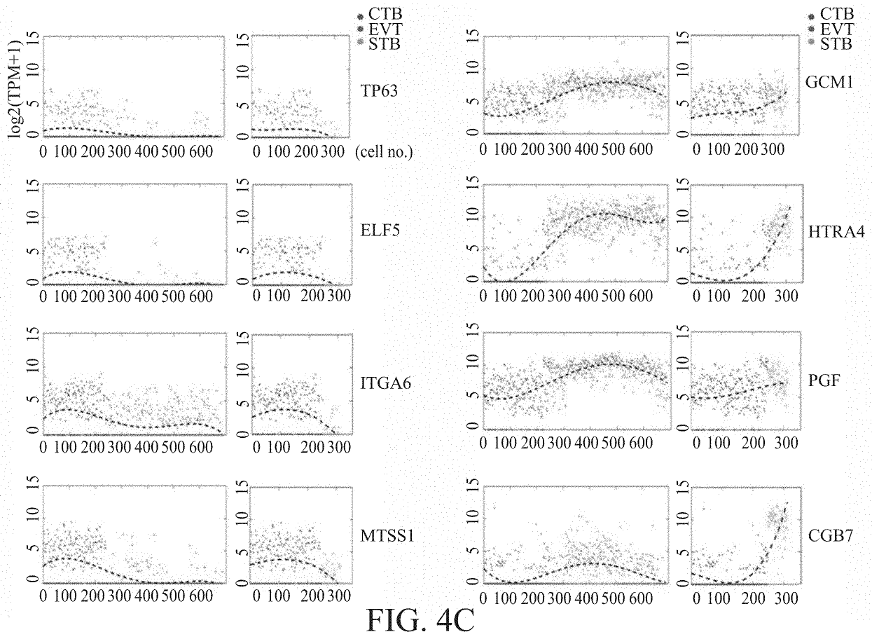

Meta-analysis of the datasets from single-cell RNA sequencing (scRNA-seq) of human first-trimester placentas and RNA-seq of TS^{CT} and TS^{blast} cells and their derivative STBs and EVTs were carried out for investigating the expression patterns of trophoblast differentiation and sternness genes. It was found that expression of ΔNp63α and trophoblast sternness genes was mutually exclusive to that of GCM1 and its target genes in TS cells and differentiated trophoblasts, supporting antagonism between GCM1 and ΔNp63α controlling trophoblast stemness, as shown in FIGS. 4A to 4C.

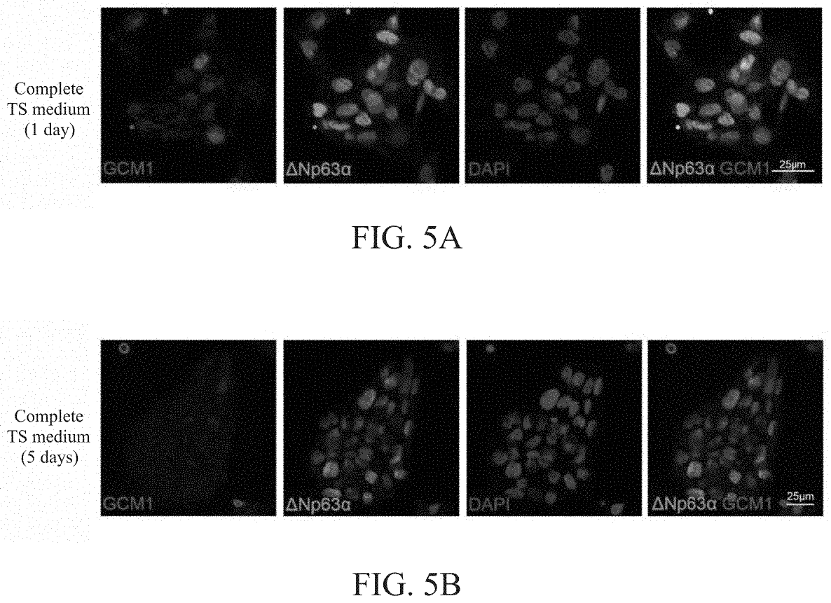

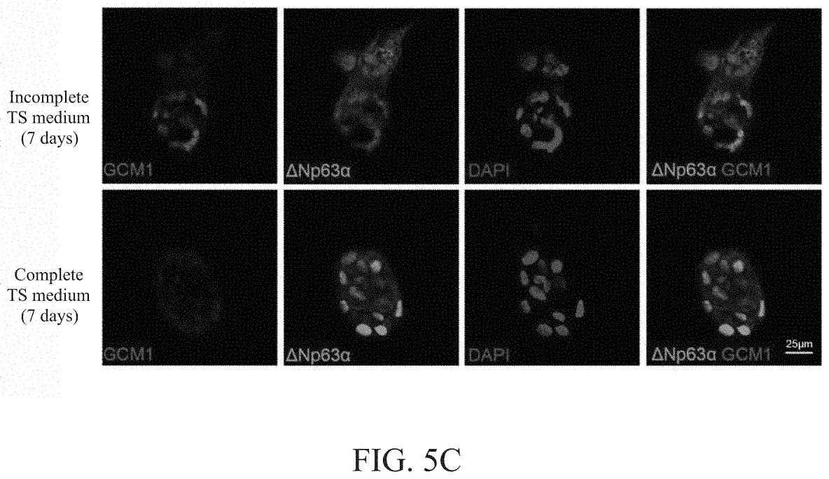

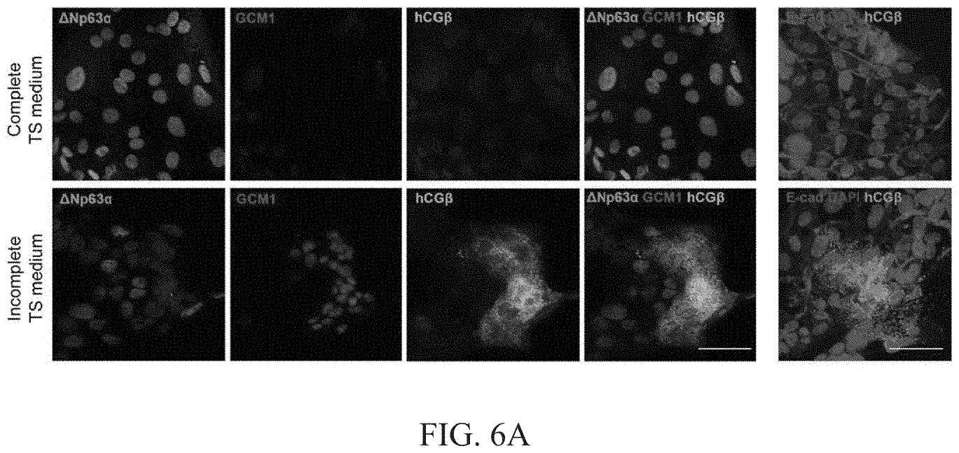

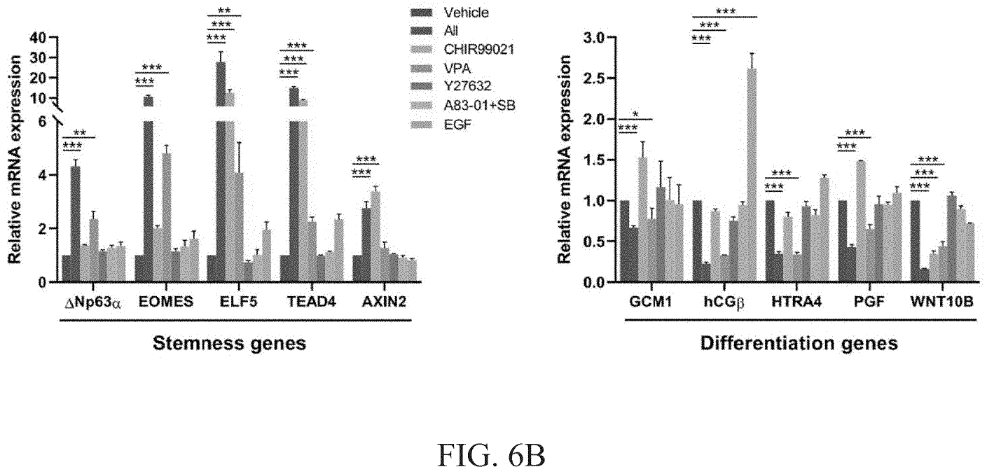

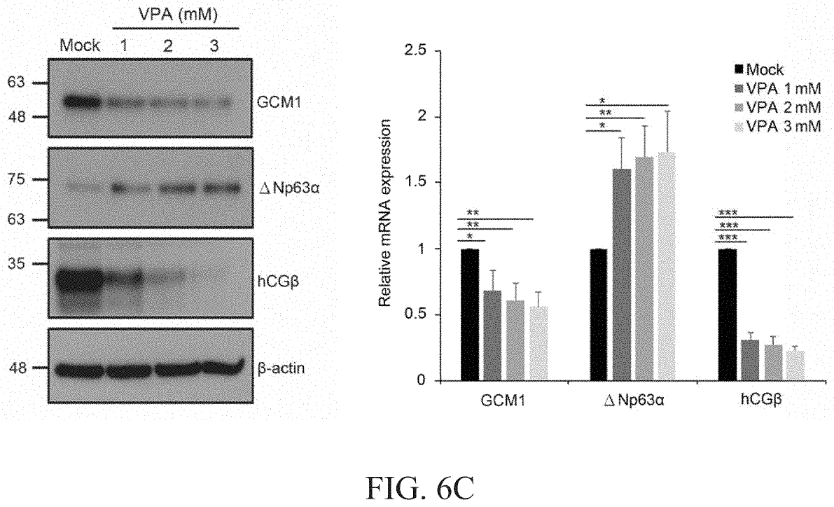

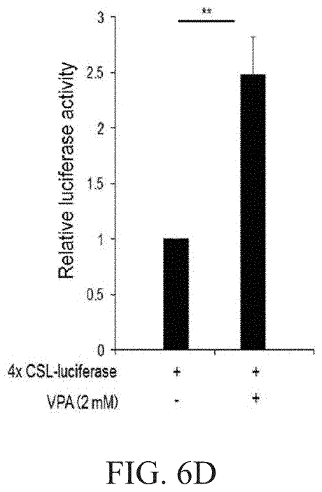

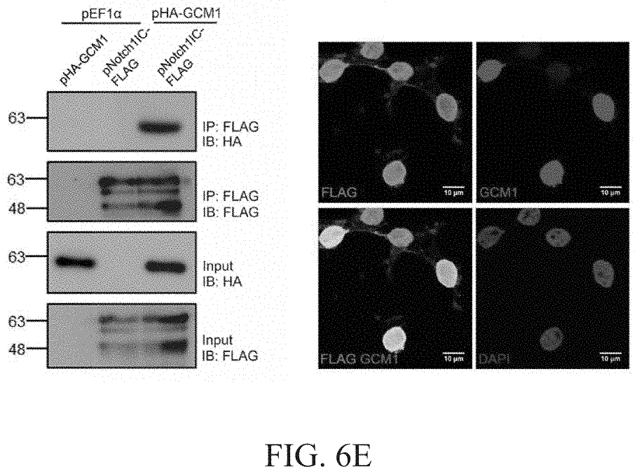

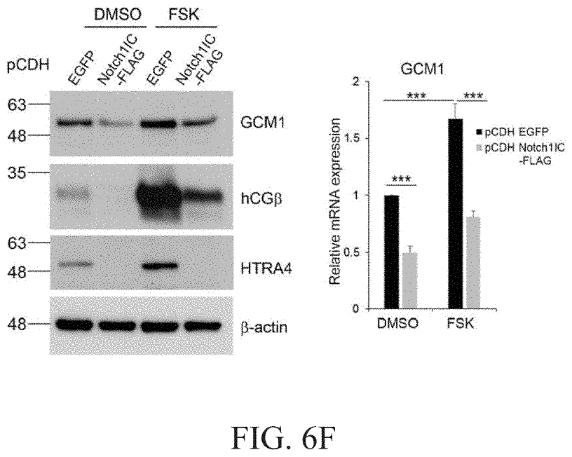

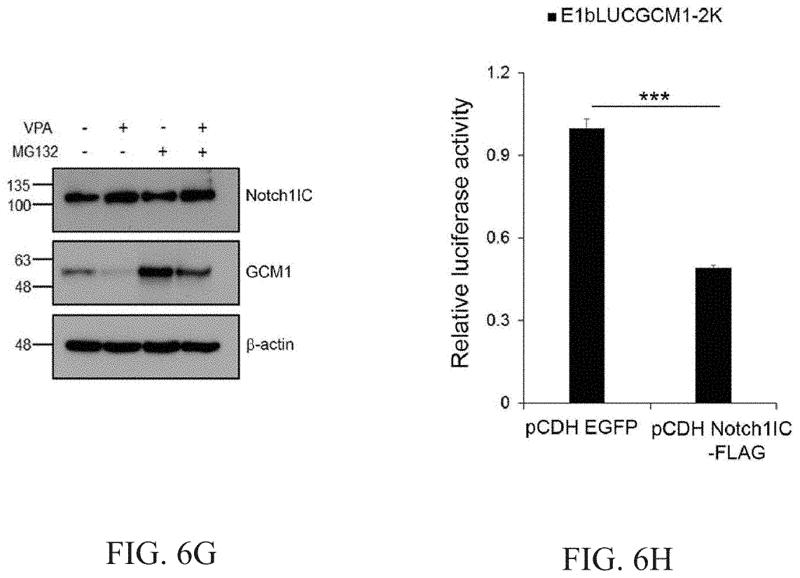

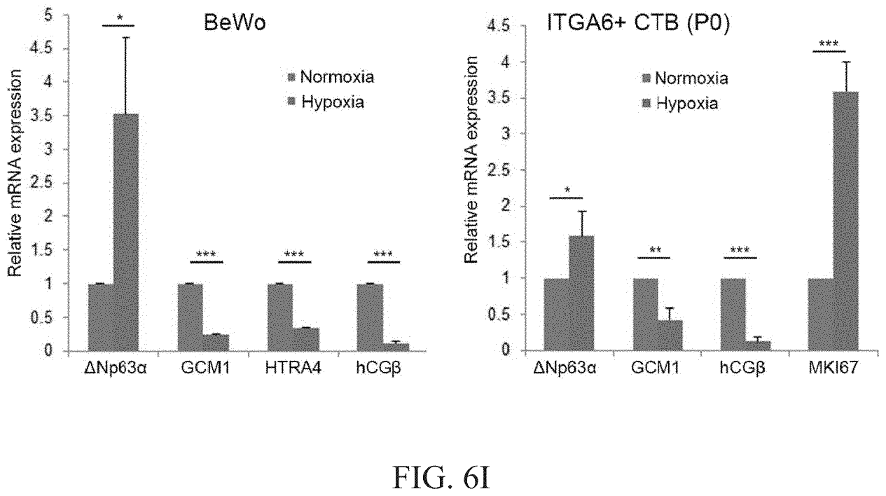

A combination of EGF and chemical inhibitors CHIR99021, A83-01, SB431542, VPA, and Y2763 were shown to facilitate the establishment of TS^{CT} and TS^{blast} cells. The effects of this combination treatment (complete TS medium) on GCM1 and ΔNp63α expression in the ITGA6-positive CTBs from term placentas were tested. The initial population of ITGA6-positive CTBs was composed of cells expressing ΔNp63α and/or GCM1, which became a more homogenous population of cells expressing ΔNp63α in the presence of EGF and chemical inhibitors for 5 days, as shown in FIGS. 5A and 5B. After withdrawal from the combination treatment (incomplete TS medium) for 7 days, the pre-treated CTBs underwent differentiation in terms of GCM1 activation and ΔNp63α suppression, as shown in FIG. 5C. In line with this, co-expression of GCM1 and hCGβ was detected in the differentiated CTBs after withdrawal from EGF and chemical inhibitors, which was shown as the vehicle in FIG. 6A.

Then, the effect of individual chemical inhibitors and EGF on the expression of genes associated with trophoblast stemness or differentiation in BeWo cells was tested. As expected, the expression of trophoblast stemness genes ΔNp63α, EOMES, ELF5, and TEAD4 was increased, whereas that of trophoblast differentiation genes GCM1, hCGβ, HTRA4, PGF, and WNT10B was decreased by the combination of EGF and chemical inhibitors, as shown in “All vs. Vehicle” in FIG. 6B.

Differential effects of EGF and individual chemical inhibitors on gene expression were observed. CHIR99021 treatment led to upregulation of ELF5, TEAD4, and AXIN2 (a WNT target control) and downregulation of WNT10B. EGF treatment suppressed GCM1 and WNT10B expression, but enhanced hCGβ expression. It was noted that VPA alone imposed a similar but less potent effect than the combination of chemical inhibitors and EGF on the trophoblast stemness and differentiation genes, as shown in FIG. 6B.

### Example 6: VPA-Mediated Notch Activation Downregulates GCM1 Activity

Subsequently, VPA was shown to increase ΔNp63α expression and suppress GCM1 and hCGβ expression in BeWo cells in a dose-dependent fashion, as shown in FIG. 6C. VPA has been reported to activate Notch signaling in carcinoid cancer cells and neuroblastoma cells. Indeed, the Notch reporter plasmid p4×CSL-luciferase was transactivated in BeWo cells treated with VPA, as shown in FIG. 6D. Moreover, interaction between Notch1IC-FLAG and HA-GCM1 and nuclear co-localization of both factors were observed in transient expression experiments, supporting that Notch1IC interacts with GCM1, as shown in FIG. 6E.

The functional outcomes of GCM1-Notch1IC interaction in BeWo cells stably expressing Notch1IC-FLAG were studied and demonstrated that Notch1IC counteracts FSK-stimulated expression of GCM1, hCGβ, and HTRA4, as shown in FIG. 6F. Furthermore, the VPA-mediated downregulation of GCM1 expression was compromised by MG132, as shown in FIG. 6G. Results of cycloheximide chase assays observed no significant effect of Notch1ICFLAG on the half-life of GCM1.

Because GCM1 autoregulates its promoter activity, the E1bLUCGCM1-2K reporter construct was transfected into mock and Notch1IC-FLAG-expressing BeWo cells, and significant decrease of GCM1-upregulated promoter activity by Notch1IC-FLAG was detected, as shown in FIG. 6H. Collectively, these results suggested that VPA activates the Notch signaling pathway to downregulate GCM1 activity resulting in elevation of ΔNp63α activity and enhancement of trophoblast stemness.

### Example 7: Derivation of TS Cells from Term Placentas

Hypoxia condition is shown to suppress GCM1 expression. GCM1, HTRA4, and hCGβ were downregulated and ΔNp63α and MKI67 were upregulated in BeWo or ITGA6-positive CTBs under hypoxia as shown in FIG. 6I.

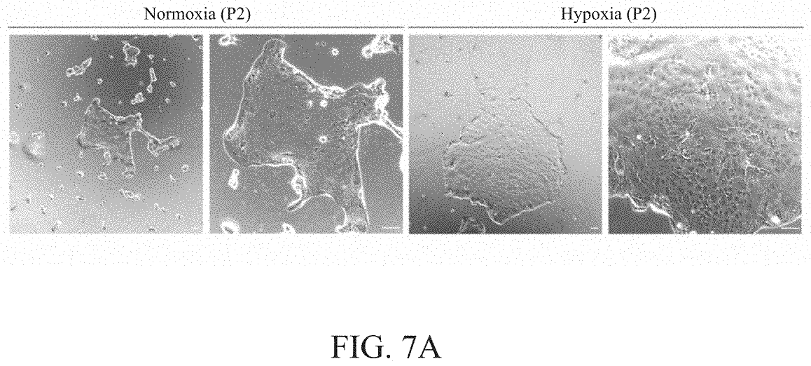

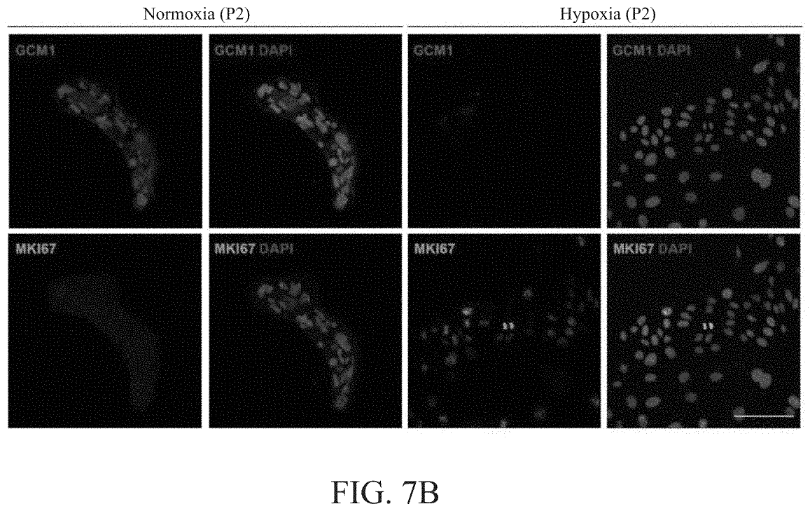

As a comparison, attempts were made to establish TS cells from term placentas by extended culture of ITGA6-positive CTBs in complete TS medium containing EGF and chemical inhibitors, but the cultured cell failed to reach the third passage under normoxia. Bright-field and immunofluorescence images indicated that the population of the second passage (P2) term ITGA6-positive CTBs under normoxia contains differentiated STBs and are GCM1-positive and MKI67-negative, as shown in FIGS. 7A and 7B. Therefore, the combination of EGF and chemical inhibitors is not sufficient to repress GCM1 activity in order to maintain trophoblast sternness.

To abolish GCM1 expression, ITGA6-positive CTBs were cultured in complete TS medium with EGF and chemical inhibitors under hypoxia. This rendered the population of P2 term CTBs to be GCM1-negative and MKI67-positive with undifferentiated morphology, as shown in FIGS. 7A and 7B. The ITGA6-positive term CTBs were maintained cultured for over 30 passages and were named as TS^{Term} cells.

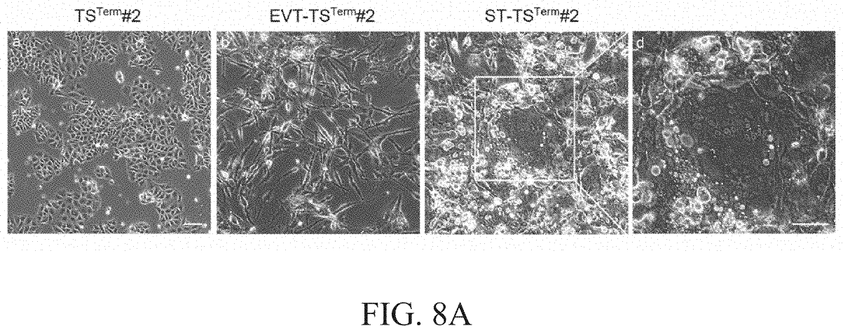

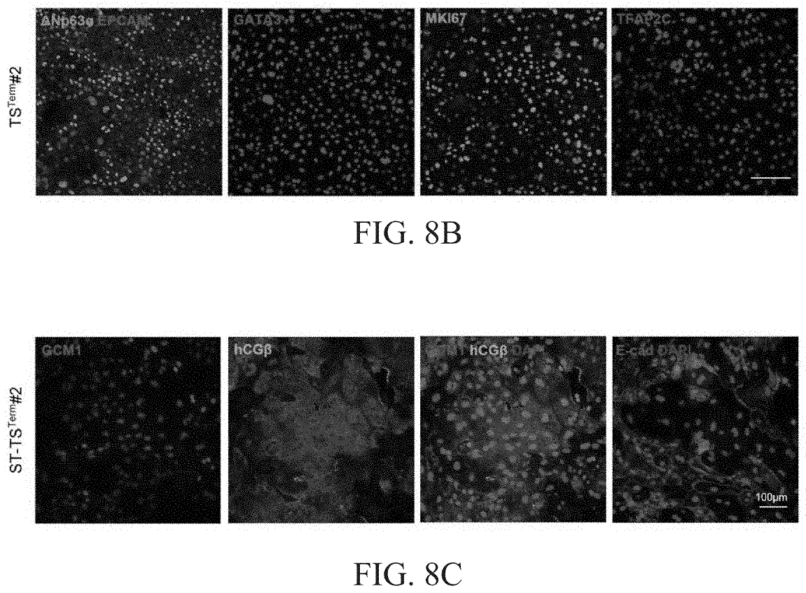

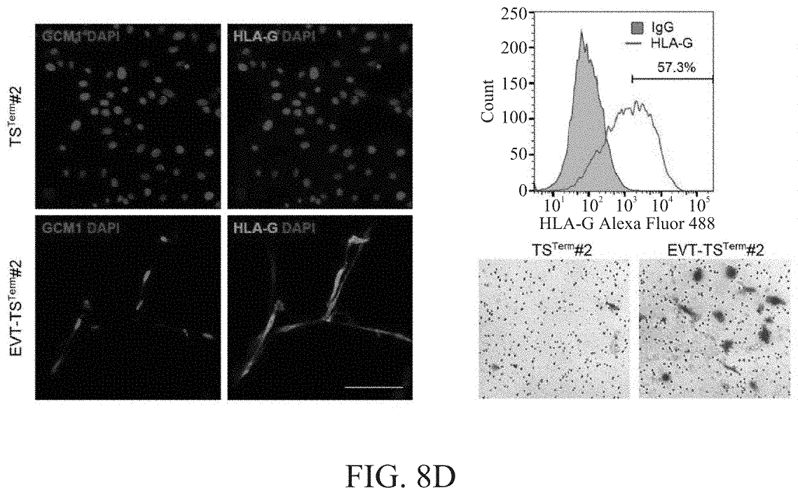

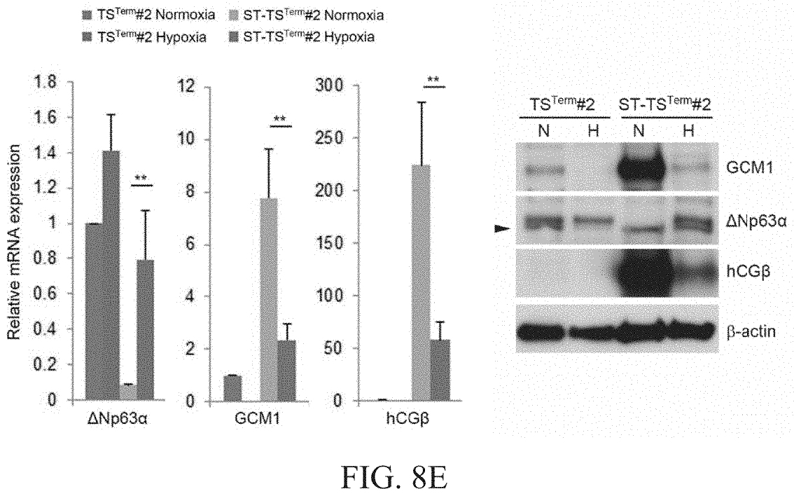

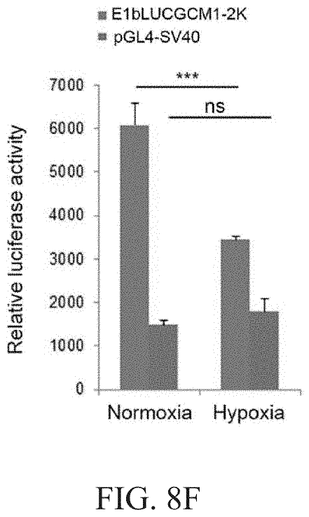

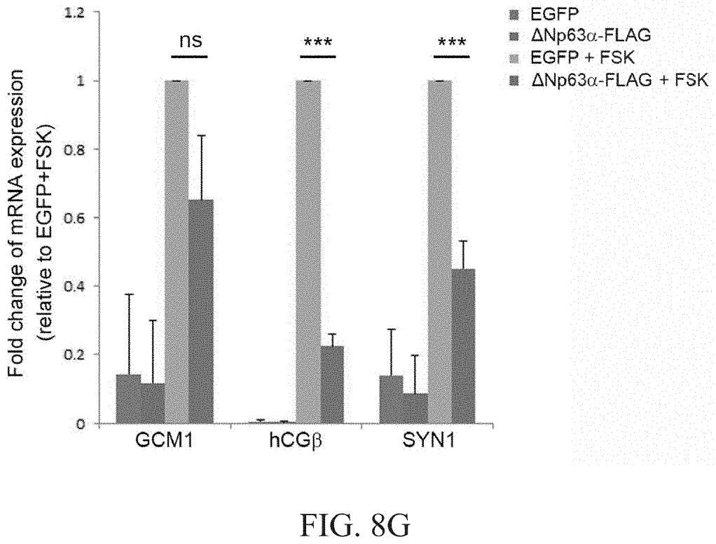

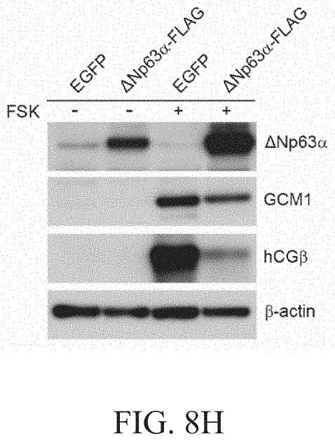

The TS^{Term} cells were shown to express ΔNp63α, EPCAM, GATA3, TFAP2, and MKI67, as depicted in FIGS. 8A and 8B. The TS^{Term} cells have bipotential ability to differentiate into multinucleated and hCGβ-positive STBs (ST-TS^{Term}) or HLA-G-positive and migratory EVTs (EVT-TS^{Term}) in response to FSK or A83-01 and NRG1 under normoxia, as shown in FIGS. 8C and 8D.

The effects of hypoxia on GCM1 activity in TS^{Term}#2 cells was confirmed by the observation that expression of GCM1 and hCGβ is significantly diminished in the hypoxic TS^{Term}#2 or ST-TS^{Term}#2 cells compared with their normoxic counterparts, as shown in FIG. 8E. Likewise, activation of the GCM1 promoter reporter plasmid E1bLUCGCM1-2K was decreased in the hypoxic TS^{Term}#2 cells, as shown in FIG. 8F. Reciprocal regulation of ΔNp63α and GCM1 activities was also tested in TS^{Term}#2 cells transduced with lentivirus harboring an empty or ΔNp63α-FLAG expression cassette. The sorted EGFP-positive mock or ΔNp63α-FLAG-expressing TS^{Term}#2 cells were treated with FSK for STB differentiation. The expression of GCM1, hCGβ, and SYN1 and therefore STB differentiation were compromised in the FSK-treated TS^{Term}#2 cells expressing ΔNp63α-FLAG, as shown in FIGS. 8G and 8H. Taken together, these results suggested that suppression of GCM1 autoregulation by hypoxia and thereby ΔNp63α upregulation involves in derivation of TS cells from term placentas.

### Example 8: Characterization of TS^{Term} Cells

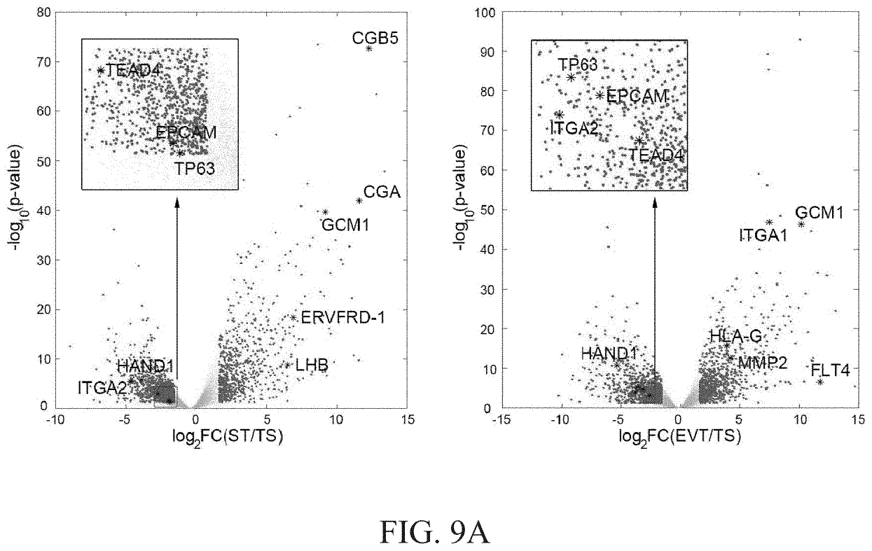

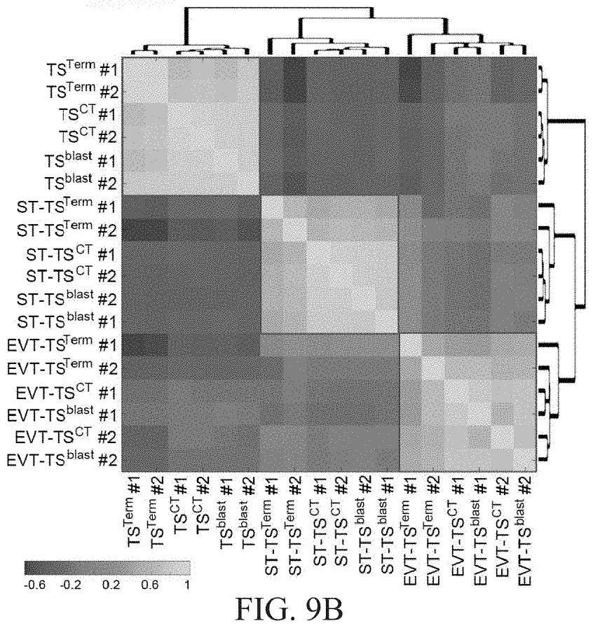

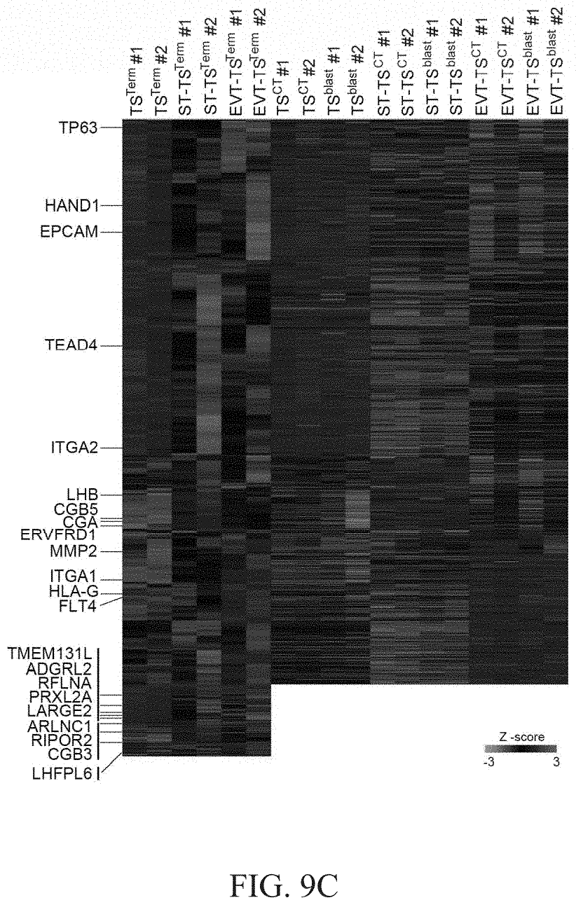

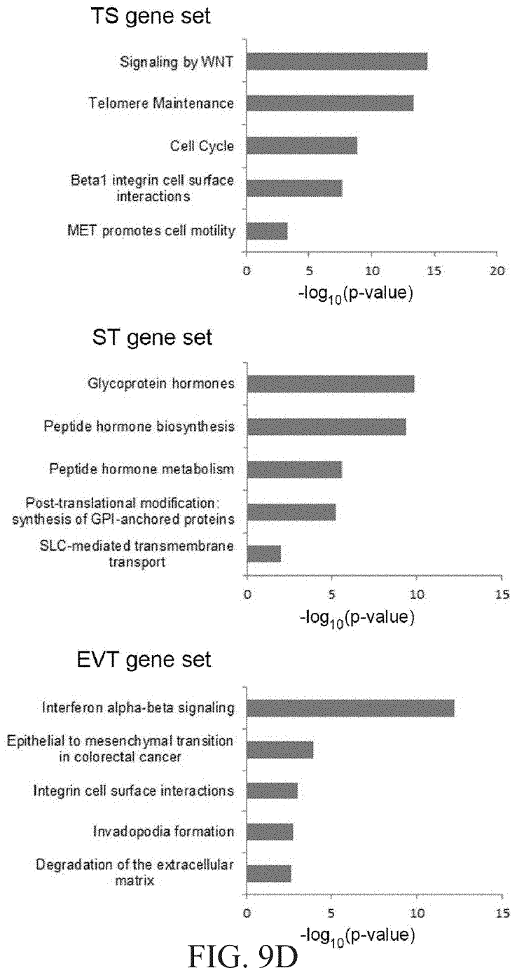

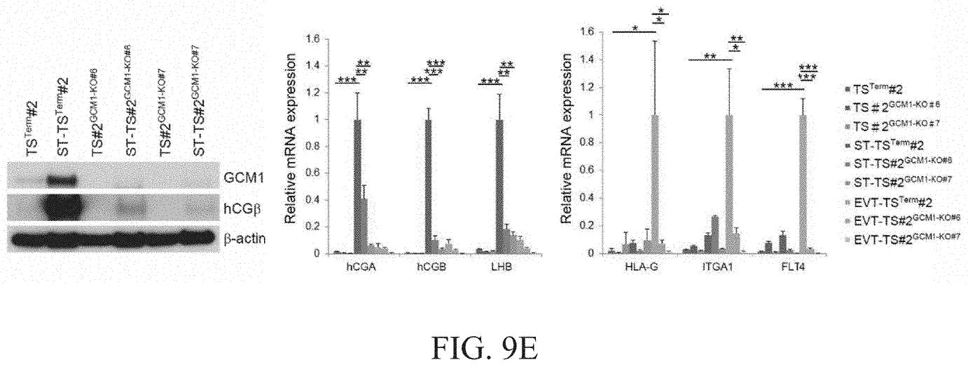

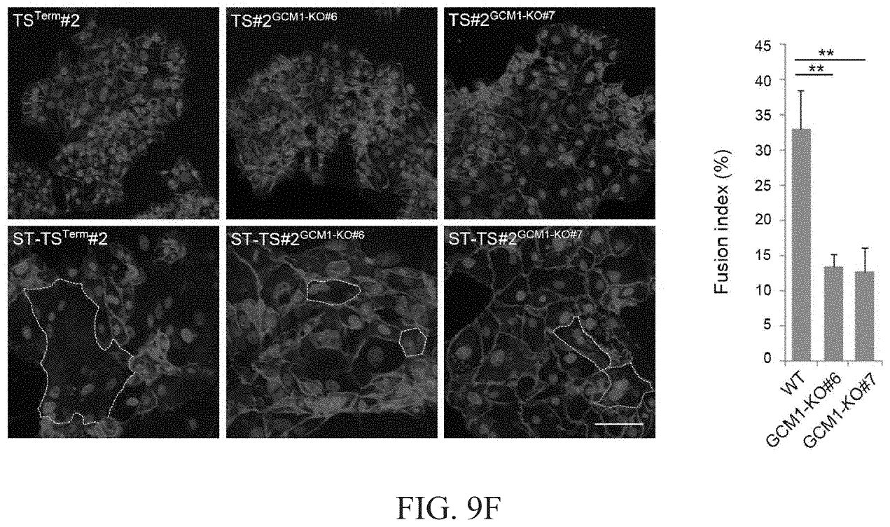

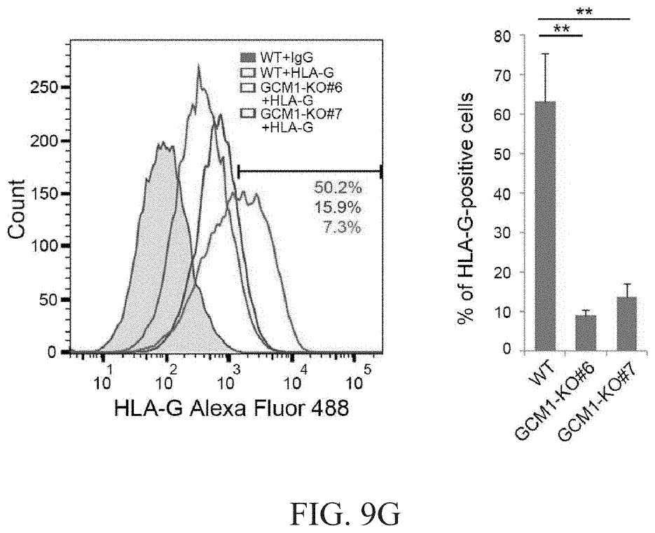

Two TS^{Term} cell lines (#1 and #2) and their derivative STBs (ST-TS^{Term}#1 and #2) and EVTs (EVT-TS^{Term}#1 and #2) were subjected to RNA sequencing analyses. Examination of the transcriptomic signatures of the different trophoblast lineages identified 2,594 genes that were differentially expressed (absolute fold change >3, p<0.05) between ST-TS^{Term} and TS^{Term} cells and 2,234 genes between EVT-TS^{Term} and TS^{Term} cells. Volcano plots of differentially expressed genes (DEGs) between differentiated trophoblasts and TS^{Term} cells revealed significantly upregulated genes in different trophoblast lineages, e.g., TEAD4, EPCAM, TP63, HAND1, and ITGA2 in TS^{Term} cells; LHB, ERVFRD-1, GCM1, CGA, and CGB5 in ST-TS^{Term} cells; and HLAG, MMP2, ITGA1, and FLT4 in EVT-TS^{Term} cells, as shown in FIG. 9A. The two groups of DEGs were merged, and a total of 3,386 genes were identified as differentiation-related genes in STBs and EVTs.

To study whether TS^{Term} cells share similar molecular signatures with TS^{CT} and TS^{blast} cells, the correlation of DEGs across the three types of TS cells and their differentiated STBs and EVTs were measured using the Pearson correlation coefficient. The result showed that TS^{Term} cells cluster most closely amongst themselves and are highly correlated with TS^{CT} and TS^{blast} cells, as shown in FIG. 9B. A total of 754, 194, and 343 genes that were predominantly expressed in TS^{Term}, ST-TS^{Term}, and EVT-TS^{Term} cells, respectively (fold change >3, p<0.05) were identified. Most of these lineage-specific genes exhibited similar expression patterns in TS^{CT} and TS^{blast} cells and their derivative STBs and EVTs. For instance, TP63, TEAD4, and ITGA2 were included in all the TS^{Term}, TS^{CT}, and TS^{blast} gene lists, CGB5, LHB, and ERVFRD1 in all the ST-TS gene lists, and HLA-G, FLT4, and MMP2 in all the EVT-TS gene lists, as shown in FIG. 9C. Of the unmatched genes, many of them, such as TMEM131, ADGRL2, RFLNA, PRXL2A, and LARGE2 in the TS^{Term} gene list, ARLNC1, RIPOR2, and CGB3 in the ST-TS^{Term} gene list, and LHFPL6 in the EVT-TS^{Term} gene list, are expressed in the CTB, STB, and EVT lineages derived from 3D-cultured human blastocysts by scRNAseq analysis, as shown in FIG. 9C. The TS^{Term} cells were further characterized by the expression of genes listed in Table 3 below.

TABLE 3
Genes expressed by TS^{Term} cells
AP003390.1
AC015522.1
AL162231.1
SMIM10L2B
AC007342.5
TMEM269
NECTIN1
JPT1
SNX18P12
MAB21L4
OR5H5P
BAIAP2-DT
AC093512.2
AC027307.2
JCAD
AC018629.1
AC007342.4
AC119751.3
AC019069.1
FAM241A
CD24
ADGRG5
AL160162.1
RIPOR1
AC090204.1
AC118754.1
GASK1B
AC241377.3
AL390719.1
MEIOC
AC016642.1
AC125603.2
CR381653.2
FAM198B-AS1
SDHAF3
RESF1
AP000439.3
AC005562.1
PCNX2
AL390334.1
SNHG19
TKFC
PCLAF
AC125807.2
AC112178.1
AC104447.1
AP001505.1
MELTF
AL031777.3
AC084759.3
AC245595.1
RPL7AP28
LINC02331
FYB2
MAP3K21
LINC02009
AC245014.3
Z93241.1
AL158839.1
TMSB15B_1
CD99
ANOS1
AL512274.1
AC008753.2
RNU2-63P
HIST2H2BB
CAVIN3

Functional annotation of the gene lists using ConsensusPathDB was carried out, discovering pathways relating to WNT, telomere maintenance, and the cell cycle that may contribute to stem cell self-renewal and proliferation assigning to the TS^{Term} gene list. Genes related to glycoprotein hormone and peptide hormone biosynthesis and metabolism were overexpressed in the ST-TS^{Term} cells. Epithelial to mesenchymal transition and integrin cell surface interaction pathways required for cell migration and invasion were upregulated in the EVT-TS^{Term} cells, as shown in FIG. 9D.

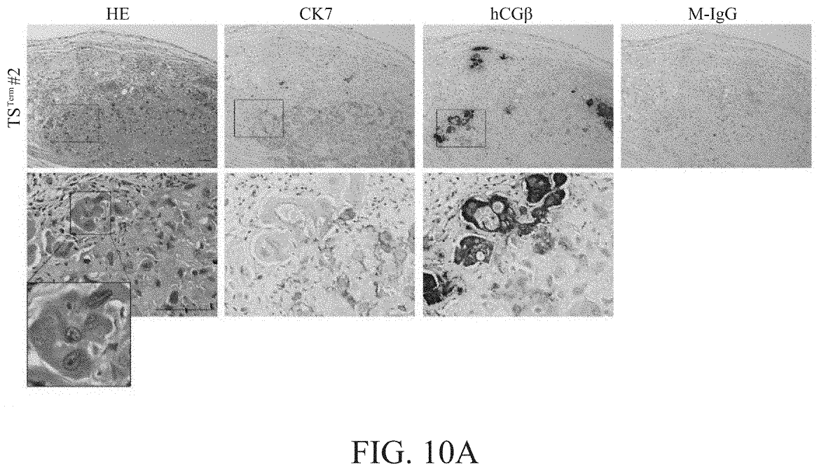

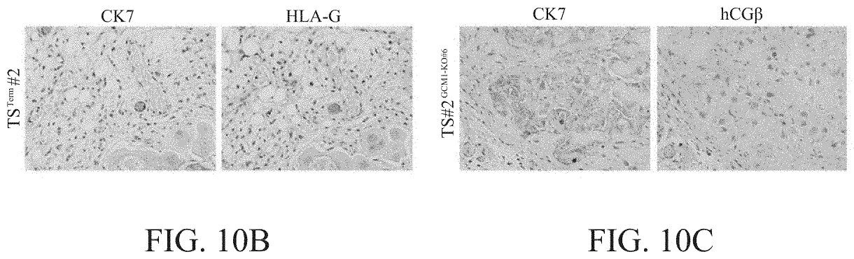

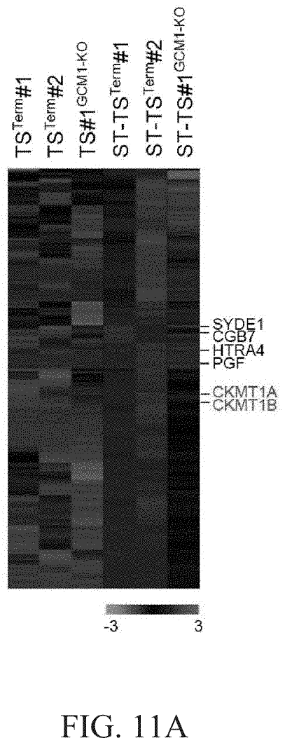

Further, TS^{Term} cells were subcutaneously injected into immunodeficient NOD-SCID mice to assess the in vivo differentiation potential of TS^{Term} cells. Biopsies of lesions formed by injected cells were collected on day 10 for immunostaining of CK7, hCGβ, and HLA-G. CK7-positive cells were readily detected in lesions, and some of them expressed hCGβ or HLA-G, suggesting that TS^{Term} cells are bipotential in vivo, as depicted in FIGS. 10A and 10B.

### Example 9: Regulation of STB Differentiation by Creatine Kinase

The roles of GCM1 in the differentiation of TS^{Term} into STBs and EVTs were investigated by knocking out GCM1 in TS^{Term}#2 cells by the CRISPR/Cas9 system, and two GCM1-KO clones TS#2^{GCM1-KO#6} and TS#2^{GCM1-KO#7} cells were generated. The bipotential differentiation capacity in TS#2^{GCM1-KO#6} and TS#2^{GCM1-KO#7} cells was eliminated in terms of STB and EVT differentiation genes as shown in FIG. 9E, cell fusion as shown in FIG. 9F, and surface expression of HLA-G as shown in FIG. 9G. These results supported that GCM1 is a regulator of STB and EVT differentiation. Correspondingly, biopsies of the lesions formed by the subcutaneously-injected TS#2^{GCM1-KO#6} cells in NOD-SCID mice exhibited expression of CK7, but not hCGβ, as shown in FIG. 10C.

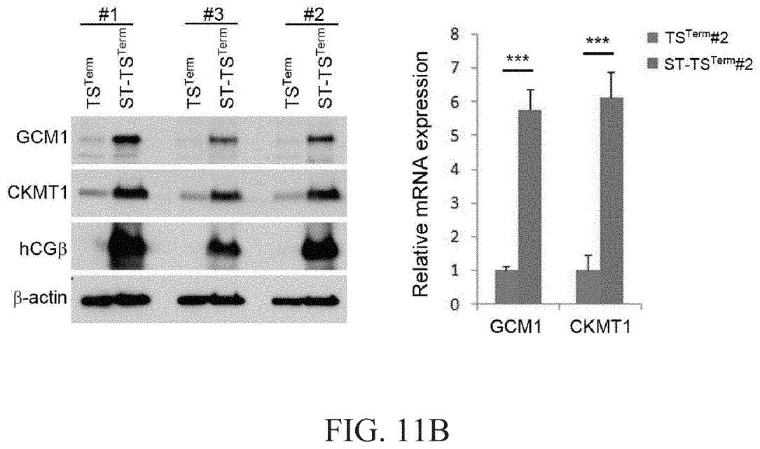

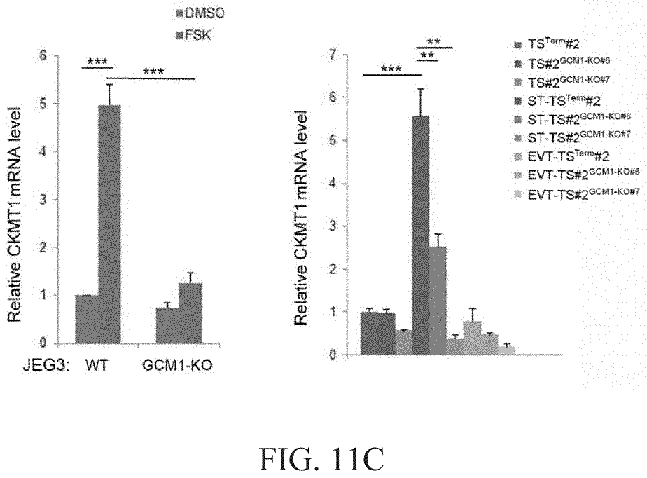

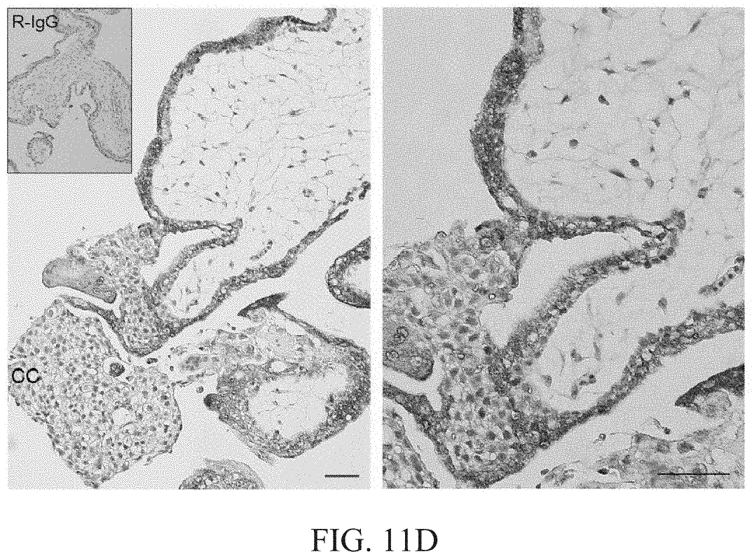

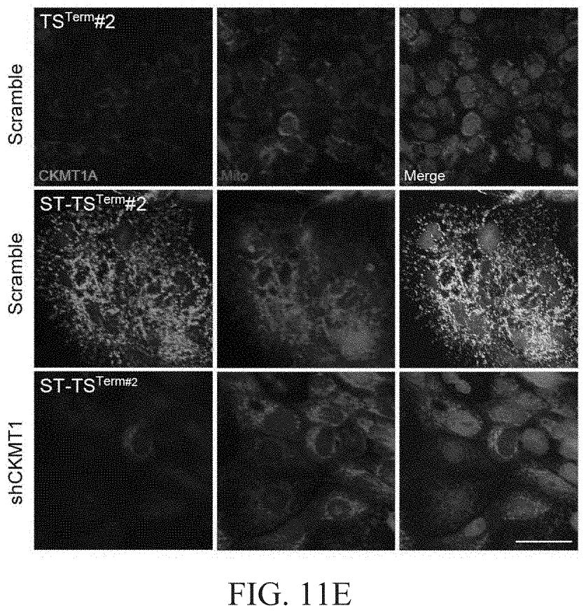

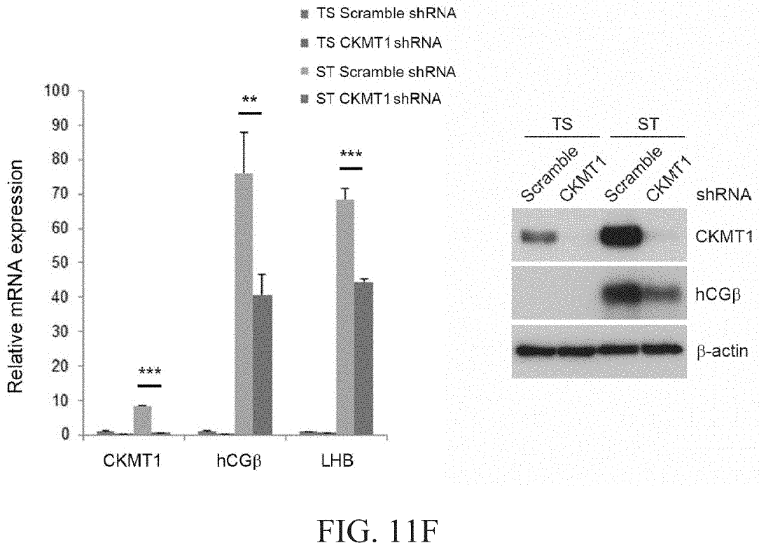

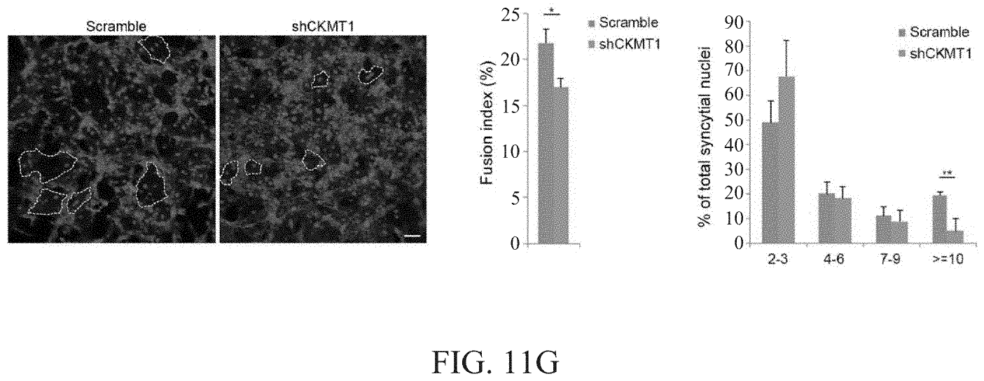

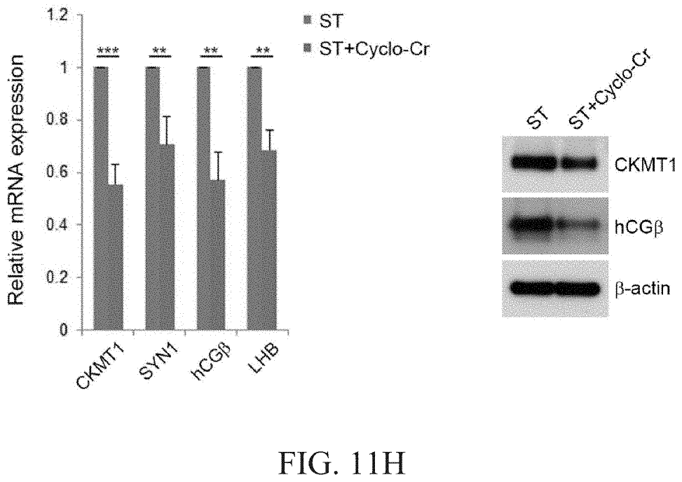

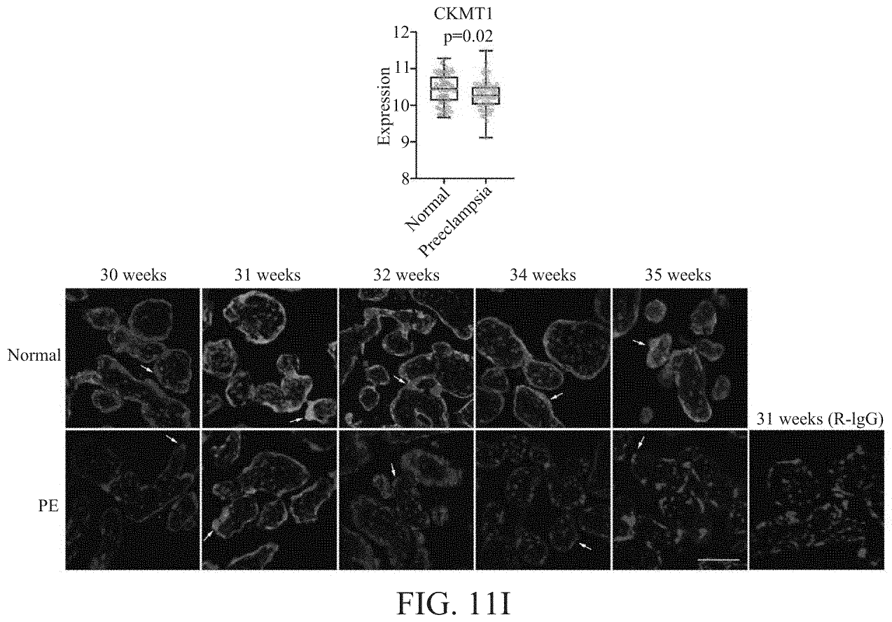

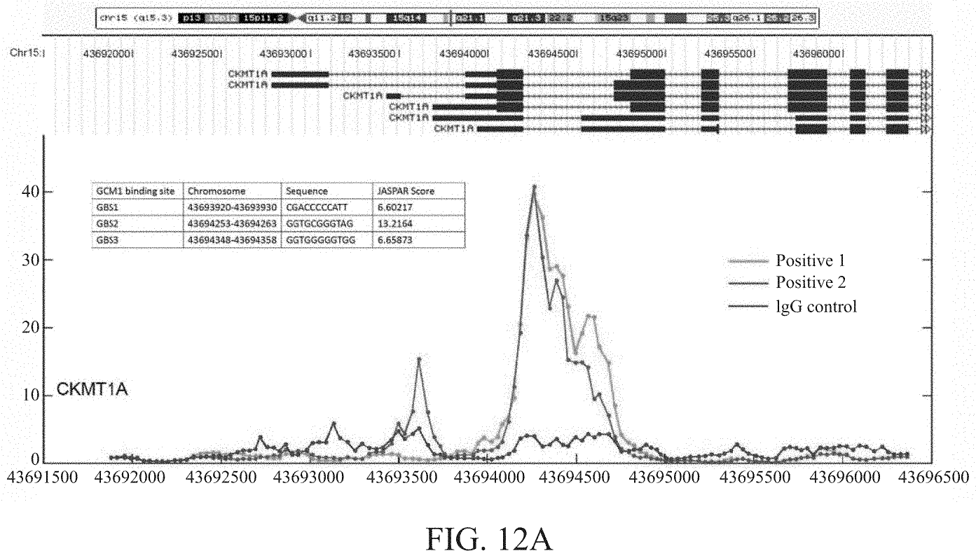

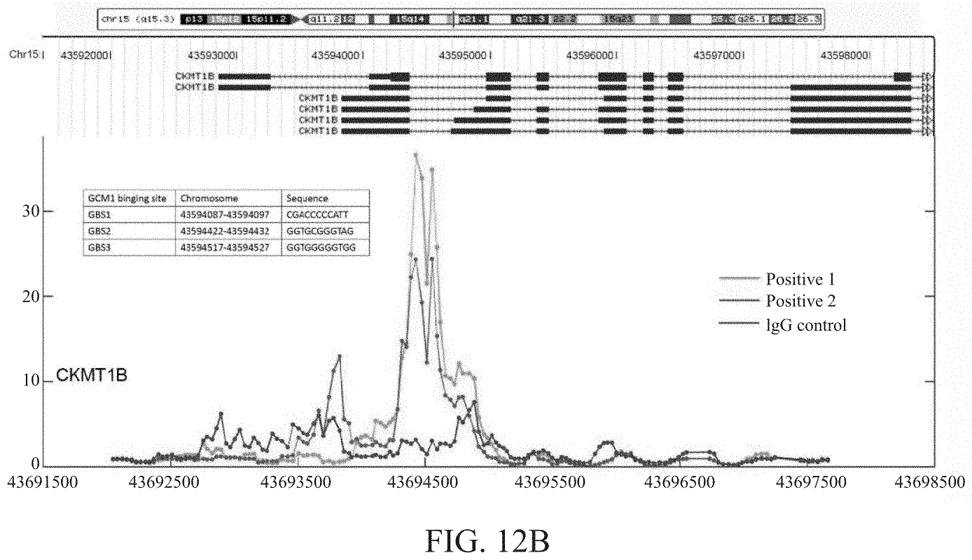

Further, RNA-seq analysis of the STBs derived from TS^{Term}#1 and TS#1^{GCM1-KO} cells was performed. GCM1 target genes mitochondrial creatine kinase 1A and 1B (CKMT1A and CKMT1B) were identified. These encode identical mitochondrial creatine kinase proteins, and catalyze the transfer of the phosphate group of ATP to the guanidino group of creatine to produce phosphocreatine, as shown in FIG. 11A and FIGS. 12A and 12B.

It was shown that CKMT1 expression was upregulated in differentiated STBs from TS^{Term}#1, -#2, and -#3 cells and FSK-treated JEG3 cells in a GCM1-dependent fashion, as this upregulation was compromised in the GCM1-KO TS^{Term} and JEG3 cells as shown in FIGS. 11B and 11C. It was shown that CKMT1 expression was not significantly changed in EVT-TS^{Term}#2 cells, suggesting that CKMT1 is not involved in EVT differentiation as shown in FIG. 11C.

In addition, immunohistochemistry results indicated that CKMT1 is primarily expressed in the STB layer, but not the subjacent CTBs, as shown in FIG. 11D.

To study the role of CKMT1 in STB differentiation, it is shown that CKMT1 is upregulated in the mitochondria of scramble control ST-TS^{Term}#2 cells compared with scramble control TS^{Term}#2 cells, as shown in FIG. 11E. STB differentiation was impaired in the CKMT1-knockdown TS^{Term}#2 cells by decreasing expression of hCGβ and LHB as well as cell fusion efficiency, as shown in FIGS. 11F and 11G.

The CKMT1 inhibitor cyclocreatine also suppressed hCGβ, SYN1, and LHB expression in the ST-TS^{Term}#2 cells, as shown in FIG. 11H. Therefore, the creatine phosphate shuttle system involves in the differentiation of STBs from TS^{Term} cells.

Defective trophoblast differentiation is associated with the pregnancy disorder preeclampsia (PE). The microarray data of control and preeclamptic placentas in public databases (GSE75010, 80 PE vs. 77 control women) were examined for CKMT1 expression. Significant decrease of CKMT1 expression was noted in PE patients, which was further confirmed by immunofluorescence microscopy of CKMT1 in the preeclamptic STBs compared with the gestational age-matched normal STBs, as shown in FIG. 11I.

While some of the embodiments of the present disclosure have been described in detail in the above, it is, however, possible for those of ordinary skill in the art to make various modifications and changes to the embodiments shown without substantially departing from the teaching and advantages of the present disclosure. Such modifications and changes are encompassed in the scope of the present disclosure as set forth in the appended claims.

### REFERENCES

1. Chiu Y H, Chen H. GATA3 inhibits GCM1 activity and trophoblast cell invasion. Sci. Rep. 2016 Feb. 22; 6:21630.
2. Chang C W, Chuang H C, Yu C, Yao T P, Chen H. Stimulation of GCMa transcriptional activity by cyclic AMP/protein kinase A signaling is attributed to CBP-mediated acetylation of GCMa. Mol. Cell Biol. 2005 October; 25(19):8401-14.
3. Chiang M H, Liang F Y, Chen C P, Chang C W, Cheong M L, Wang L J, Liang C Y, Lin F Y, Chou C C, Chen H. Mechanism of hypoxia-induced GCM1 degradation: implications for the pathogenesis of preeclampsia. J. Biol. Chem. 2009 Jun. 26; 284(26):17411-9.
4. Wang L J, Cheong M L, Lee Y S, Lee M T, Chen H. High-temperature requirement protein A4 (HtrA4) suppresses the fusogenic activity of syncytin-1 and promotes trophoblast invasion. Mol. Cell Biol. 2012 September; 32(18):3707-17.
5. Chen H, Chong Y, Liu C L. Active intracellular domain of Notch enhances transcriptional activation of CCAAT/enhancer binding protein beta on a rat pregnancy-specific glycoprotein gene. Biochemistry. 2000 Feb. 22; 39(7):1675-82.
6. Okae H, Toh H, Sato T, Hiura H, Takahashi S, Shirane K, Kabayama Y, Suyama M, Sasaki H, Arima T. Derivation of Human Trophoblast Stem Cells. Cell Stem Cell. 2018 Jan. 4; 22(1):50-63.e6.
7. Cheong M L, Wang L J, Chuang P Y, Chang C W, Lee Y S, Lo H F, Tsai M S, Chen H. A Positive Feedback Loop between Glial Cells Missing 1 and Human Chorionic Gonadotropin (hCG) Regulates Placental hCGβ Expression and Cell Differentiation. Mol. Cell Biol. 2015 Oct. 26; 36(1):197-209.

What is claimed is:

1. A pluripotent trophoblast stem cell preparation prepared from cells obtained from a term placenta.

2. The pluripotent trophoblast cell preparation according to claim 1, wherein the cells obtained from the term placenta are cytotrophoblasts.

3. The pluripotent trophoblast cell preparation according to claim 2, wherein the cytotrophoblasts are ITGA6-positive cytotrophoblasts.

4. The pluripotent trophoblast cell preparation according to claim 1, which is capable of indefinite proliferation in vitro in an undifferentiated state.

5. The pluripotent trophoblast cell preparation according to claim 4, which is maintained at the undifferentiated state by manipulating a level of at least one of glial cells missing 1 (GCM1) and ΔNp63α or a combination thereof.

6. The pluripotent trophoblast cell preparation according to claim 5, wherein the level of GCM1 is suppressed.

7. The pluripotent trophoblast cell preparation according to claim 6, wherein the level of ΔNp63α is enhanced.

8. The pluripotent trophoblast cell preparation according to claim 4, which is maintained for at least 30 passages.

9. The pluripotent trophoblast cell preparation according to claim 1, which is capable of differentiation.

10. The pluripotent trophoblast cell preparation according to claim 1, which is capable of differentiation into cells of a trophoblast lineage in vitro.

11. The pluripotent trophoblast cell preparation according to claim 1, which is capable of differentiation into cells of a trophoblast lineage in vivo.

12. The pluripotent trophoblast cell preparation according to claim 9, which is capable of differentiation into multinucleated syncytiotrophoblasts (STBs) or invasive extravillous trophoblasts (EVTs).

13. The pluripotent trophoblast cell preparation according to claim 1, which is characterized by expression of at least one of TP63, TEAD4, EPCAM, HAND1 and ITGA2.

14. A method for establishing the pluripotent trophoblast cell preparation according to claim 1, comprising:
obtaining the term placenta from a subject;
isolating placental cytotrophoblasts from the term placenta; and
manipulating a level of at least one of glial cells missing 1 (GCM1) and ΔNp63α or a combination thereof in the isolated placental cytotrophoblasts.

15. The method according to claim 14, further comprising culturing the placental cytotrophoblasts under a hypoxia condition.

16. A method for establishing a disease model for a pregnancy related disorder, comprising:
manipulating a target gene in the pluripotent trophoblast cell preparation according to claim 1; and
analyzing a level of a gene in the pluripotent trophoblast cell preparation.

17. The method according to claim 16, wherein:
manipulating the target gene includes eliminating expression of glial cells missing 1 (GCM1) in the pluripotent trophoblast cell preparation; and
the method further comprises, after analyzing a level of a gene in the pluripotent trophoblast cell preparation, identifying a gene having a different level.

18. The method according to claim 16, wherein the pregnancy related disorder is preeclampsia.

19. A method for diagnosing preeclampsia in a subject in need thereof, comprising:
obtaining a biological sample from the subject;
determining a level of mitochondrial creatine kinase 1 (CKMT1) in the biological sample;
comparing the level of CKMT1 with a predetermined level; and
determining the subject to be suffering from preeclampsia as the level of CKMT1 is lower than the predetermined level.


SEQUENCE LISTING (48 sequences)

SEQ ID NO: 1 - DNA, 7201 bp, Artificial Sequence
  plasmid construct
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttatgttgtacctggaaaacaatgcccagactcaatttagtgagccacagtacacgaacctggggctcctgaacagcatggaccagcagattcagaacggctcctcgtccaccagtccctataacacagaccacgcgcagaacagcgtcacggcgccctcgccctacgcacagcccagctccaccttcgatgctctctctccatcacccgccatcccctccaacaccgactacccaggcccgcacagtttcgacgtgtccttccagcagtcgagcaccgccaagtcggccacctggacgtattccactgaactgaagaaactctactgccaaattgcaaagacatgccccatccagatcaaggtgatgaccccacctcctcagggagctgttatccgcgccatgcctgtctacaaaaaagctgagcacgtcacggaggtggtgaagcggtgccccaaccatgagctgagccgtgaattcaacgagggacagattgcccctcctagtcatttgattcgagtagaggggaacagccatgcccagtatgtagaagatcccatcacaggaagacagagtgtgctggtaccttatgagccaccccaggttggcactgaattcacgacagtcttgtacaatttcatgtgtaacagcagttgtgttggagggatgaaccgccgtccaattttaatcattgttactctggaaaccagagatgggcaagtcctgggccgacgctgctttgaggcccggatctgtgcttgcccaggaagagacaggaaggcggatgaagatagcatcagaaagcagcaagtttcggacagtacaaagaacggtgatggtacgaagcgcccgtttcgtcagaacacacatggtatccagatgacatccatcaagaaacgaagatccccagatgatgaactgttatacttaccagtgaggggccgtgagacttatgaaatgctgttgaagatcaaagagtccctggaactcatgcagtaccttcctcagcacacaattgaaacgtacaggcaacagcaacagcagcagcaccagcacttacttcagaaacagacctcaatacagtctccatcttcatatggtaacagctccccacctctgaacaaaatgaacagcatgaacaagctgccttctgtgagccagcttatcaaccctcagcagcgcaacgccctcactcctacaaccattcctgatggcatgggagccaacattcccatgatgggcacccacatgccaatggctggagacatgaatggactcagccccacccaggcactccctcccccactctccatgccatccacctcccactgcacacccccacctccgtatcccacagattgcagcattgtcagtttcttagcgaggttgggctgttcatcatgtctggactatttcacgacccaggggctgaccaccatctatcagattgagcattactccatggatgatctggcaagtctgaaaatccctgagcaatttcgacatgcgatctggaagggcatcctggaccaccggcagctccacgaattctcctccccttctcatctcctgcggaccccaagcagtgcctctacagtcagtgtgggctccagtgagacccggggtgagcgtgttattgatgctgtgcgattcaccctccgccagaccatctctttcccaccccgagatgagtggaatgacttcaactttgacatggatgctcgccgcaataagcaacagcgcatcaaagaggagggggagggggattataaagatgatgatgataaaggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 2 - DNA, 7210 bp, Artificial Sequence
  plasmid construct
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttatgttgtacctggaaaacaatgcccagactcaatttagtgagccacagtacacgaacctggggctcctgaacagcatggaccagcagattcagaacggctcctcgtccaccagtccctataacacagaccacgcgcagaacagcgtcacggcgccctcgccctacgcacagcccagctccaccttcgatgctctctctccatcacccgccatcccctccaacaccgactacccaggcccgcacagtttcgacgtgtccttccagcagtcgagcaccgccaagtcggccacctggacgtattccactgaactgaagaaactctactgccaaattgcaaagacatgccccatccagatcaaggtgatgaccccacctcctcagggagctgttatccgcgccatgcctgtctacaaaaaagctgagcacgtcacggaggtggtgaagcggtgccccaaccatgagctgagccgtgaattcaacgagggacagattgcccctcctagtcatttgattcgagtagaggggaacagccatgcccagtatgtagaagatcccatcacaggaagacagagtgtgctggtaccttatgagccaccccaggttggcactgaattcacgacagtcttgtacaatttcatgtgtaacagcagttgtgttggagggatgaaccgccgtccaattttaatcattgttactctggaaaccagagatgggcaagtcctgggccgacgctgctttgaggcccggatctgtgcttgcccaggaagagacaggaaggcggatgaagatagcatcagaaagcagcaagtttcggacagtacaaagaacggtgatggtacgaagcgcccgtttcgtcagaacacacatggtatccagatgacatccatcaagaaacgaagatccccagatgatgaactgttatacttaccagtgaggggccgtgagacttatgaaatgctgttgaagatcaaagagtccctggaactcatgcagtaccttcctcagcacacaattgaaacgtacaggcaacagcaacagcagcagcaccagcacttacttcagaaacagacctcaatacagtctccatcttcatatggtaacagctccccacctctgaacaaaatgaacagcatgaacaagctgccttctgtgagccagcttatcaaccctcagcagcgcaacgccctcactcctacaaccattcctgatggcatgggagccaacattcccatgatgggcacccacatgccaatggctggagacatgaatggactcagccccacccaggcactccctcccccactctccatgccatccacctcccactgcacacccccacctccgtatcccacagattgcagcattgtcagtttcttagcgaggttgggctgttcatcatgtctggactatttcacgacccaggggctgaccaccatctatcagattgagcattactccatggatgatctggcaagtctgaaaatccctgagcaatttcgacatgcgatctggaagggcatcctggaccaccggcagctccacgaattctcctccccttctcatctcctgcggaccccaagcagtgcctctacagtcagtgtgggctccagtgagacccggggtgagcgtgttattgatgctgtgcgattcaccctccgccagaccatctctttcccaccccgagatgagtggaatgacttcaactttgacatggatgctcgccgcaataagcaacagcgcatcaaagaggagggggagtgagggtacccttatgatgtgccagattatgcctaaggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 3 - DNA, 8207 bp, Artificial Sequence
  plasmid construct
ccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattaagcttgcggccgcgaattcatcgatagatctgatatcatgttgtacctggaaaacaatgcccagactcaatttagtgagccacagtacacgaacctggggctcctgaacagcatggaccagcagattcagaacggctcctcgtccaccagtccctataacacagaccacgcgcagaacagcgtcacggcgccctcgccctacgcacagcccagctccaccttcgatgctctctctccatcacccgccatcccctccaacaccgactacccaggcccgcacagtttcgacgtgtccttccagcagtcgagcaccgccaagtcggccacctggacgtattccactgaactgaagaaactctactgccaaattgcaaagacatgccccatccagatcaaggtgatgaccccacctcctcagggagctgttatccgcgccatgcctgtctacaaaaaagctgagcacgtcacggaggtggtgaagcggtgccccaaccatgagctgagccgtgaattcaacgagggacagattgcccctcctagtcatttgattcgagtagaggggaacagccatgcccagtatgtagaagatcccatcacaggaagacagagtgtgctggtaccttatgagccaccccaggttggcactgaattcacgacagtcttgtacaatttcatgtgtaacagcagttgtgttggagggatgaaccgccgtccaattttaatcattgttactctggaaaccagagatgggcaagtcctgggccgacgctgctttgaggcccggatctgtgcttgcccaggaagagacaggaaggcggatgaagatagcatcagaaagcagcaagtttcggacagtacaaagaacggtgatggtacgaagcgcccgtttcgtcagaacacacatggtatccagatgacatccatcaagaaacgaagatccccagtgatgaactgttatacttaccagtgaggggccgtgagacttatgaaatgctgttgaagatcaaagagtccctggaactcatgcagtaccttcctcagcacacaattgaaacgtacaggcaacagcaacagcagcagcaccagcacttacttcagaaacagacctcaatacagtctccatcttcatatggtaacagctccccacctctgaacaaaatgaacagcatgaacaagctgccttctgtgagccagcttatcaaccctcagcagcgcaacgccctcactcctacaaccattcctgatggcatgggagccaacattcccatgatgggcacccacatgccaatggctggagacatgaatggactcagccccacccaggcactccctcccccactctccatgccatccacctcccactgcacacccccacctccgtatcccacagattgcagcattgtcagtttcttagcgaggttgggctgttcatcatgtctggactatttcacgacccaggggctgaccaccatctatcagattgagcattactccatggatgatctggcaagtctgaaaatccctgagcaatttcgacatgcgatctggaagggcatcctggaccaccggcagctccacgaattctcctccccttctcatctcctgcggaccccaagcagtgcctctacagtcagtgtgggctccagtgagacccggggtgagcgtgttattgatgctgtgcgattcaccctccgccagaccatctctttcccaccccgagatgagtggaatgacttcaactttgacatggatgctcgccgcaataagcaacagcgcatcaaagaggagggggagtctagaggatcctttaaagctatggagcaaaagctcatttctgaagaggacttgaataaaatggagcaaaagctcatttctgaagaggacttgaatgaaatggagcaaaagctcatttctgaagaggacttgaatgaaatgtagagcttgggcgacctcagatcccgggctgactacaaagaccatgacggtgattataaagatcatgacatcgactacaaggatgacgatgacaagtagtgatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggaattgatcagcttgggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgggctcgatcccctcgcgagttggttcagctgctgcctgaggctggacgacctcgcggagttctaccggcagtgcaaatccgtcggcatccaggaaaccagcagcggctatccgcgcatccatgcccccgaactgcaggagtggggaggcacgatggccgctttggtcgacccggacgggacgctcctgcgcctgatacagaacgaattgcttgcaggcatctcatgagtgtgtcttcccgttttccgcctgaggtcactgcgtggatggagcgctggcgcctgctgcgcgacggcgagctgctcaccacccactcgccaagctggaaccgtaaaaaggccgcgttgctggcgtttttccataggctccgccgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaattccctatagtgagtcgtattaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaattt

SEQ ID NO: 4 - DNA, 9308 bp, Artificial Sequence
  plasmid construct
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggttaacttttaaaagaaaaggggggattggggggtacagtgcaggggaaagaatagtagacataatagcaacagacatacaaactaaagaattacaaaaacaaattacaaaaattcaaaattttatcgatactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagattctagagatgttgtacctggaaaacaatgcccagactcaatttagtgagccacagtacacgaacctggggctcctgaacagcatggaccagcagattcagaacggctcctcgtccaccagtccctataacacagaccacgcgcagaacagcgtcacggcgccctcgccctacgcacagcccagctccaccttcgatgctctctctccatcacccgccatcccctccaacaccgactacccaggcccgcacagtttcgacgtgtccttccagcagtcgagcaccgccaagtcggccacctggacgtattccactgaactgaagaaactctactgccaaattgcaaagacatgccccatccagatcaaggtgatgaccccacctcctcagggagctgttatccgcgccatgcctgtctacaaaaaagctgagcacgtcacggaggtggtgaagcggtgccccaaccatgagctgagccgtgaattcaacgagggacagattgcccctcctagtcatttgattcgagtagaggggaacagccatgcccagtatgtagaagatcccatcacaggaagacagagtgtgctggtaccttatgagccaccccaggttggcactgaattcacgacagtcttgtacaatttcatgtgtaacagcagttgtgttggagggatgaaccgccgtccaattttaatcattgttactctggaaaccagagatgggcaagtcctgggccgacgctgctttgaggcccggatctgtgcttgcccaggaagagacaggaaggcggatgaagatagcatcagaaagcagcaagtttcggacagtacaaagaacggtgatggtacgaagcgcccgtttcgtcagaacacacatggtatccagatgacatccatcaagaaacgaagatccccagtgatgaactgttatacttaccagtgaggggccgtgagacttatgaaatgctgttgaagatcaaagagtccctggaactcatgcagtaccttcctcagcacacaattgaaacgtacaggcaacagcaacagcagcagcaccagcacttacttcagaaacagacctcaatacagtctccatcttcatatggtaacagctccccacctctgaacaaaatgaacagcatgaacaagctgccttctgtgagccagcttatcaaccctcagcagcgcaacgccctcactcctacaaccattcctgatggcatgggagccaacattcccatgatgggcacccacatgccaatggctggagacatgaatggactcagccccacccaggcactccctcccccactctccatgccatccacctcccactgcacacccccacctccgtatcccacagattgcagcattgtcagtttcttagcgaggttgggctgttcatcatgtctggactatttcacgacccaggggctgaccaccatctatcagattgagcattactccatggatgatctggcaagtctgaaaatccctgagcaatttcgacatgcgatctggaagggcatcctggaccaccggcagctccacgaattctcctccccttctcatctcctgcggaccccaagcagtgcctctacagtcagtgtgggctccagtgagacccggggtgagcgtgttattgatgctgtgcgattcaccctccgccagaccatctctttcccaccccgagatgagtggaatgacttcaactttgacatggatgctcgccgcaataagcaacagcgcatcaaagaggagggggagggggattataaagatgatgatgataaaggatccgcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagacgccaccatggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatcccgcgctcagtcgtccaattctgccgtggacggcaccgccggacccggctccaccggatctcgctaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 5 - DNA, 6247 bp, Artificial Sequence
  plasmid construct
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttatgccccgcgcgttcctggtgaagaagccgtgcgtctccacgtgcaagaggaactggagcgagctccccgacgaggagcgcggcgagatctacgtgccagtcagcctgggcttctgcccaccacagccctaccgggagccggaaccctctgtggccgaacccccttcctgcccgctggctttgaacatgagccttcgagactctagctacagcatggcccccgggccctgtgtggtggcccagctgccctctgaagacatgggccacttgacagacccccagagcagagaccatggcttcctgcgcaccaagatgaaggtgacccttggggacagtcccagtggagacctgttcacctgccgtgtctgccagaaggccttcacctaccagcgcatgctgaaccgccacatgaagtgtcacaacgacgtcaagaggcacctctgcacgtactgcgggaagggcttcaatgacaccttcgacctcaagagacacgtccgaactcacactggcgtgcggccctacaagtgcagcctgtgtgacaaggccttcacgcagcgctgctctctggagtctcacctcaagaagatccatggtgtgcagcagaagtacgcgtacaaggagcggcgggccaagctgtacgtgtgtgaggagtgcggctgcacatctgagagccaggagggccacgtcctgcacctgaaggagcaccaccctgacagcccgctgctgcgcaagacctccaagaaggtggccgtggcactacagaacactgtcacttccctgctgcagggcagcccccacctgggggattataaagatgatgatgataaatgaggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 6 - DNA, 8908 bp, Artificial Sequence
  plasmid construct
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggttaacttttaaaagaaaaggggggattggggggtacagtgcaggggaaagaatagtagacataatagcaacagacatacaaactaaagaattacaaaaacaaattacaaaaattcaaaattttatcgatactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagattctagaatggtgctgctgtcccgcaagcgccggcggcagcatggccagctctggttccctgagggtttcaaagtgtcagaggccagcaagaagaagcggagagagcccctcggcgaggactcagtcggcctcaagcccctgaagaatgcctcagatggtgctctgatggacgacaatcagaacgagtggggagacgaagacctggagaccaagaagttccggtttgaggagccagtagttctccctgacctgagtgatcagactgaccacaggcagtggacccagcagcacctggacgctgctgacctgcgcatgtctgccatggccccaacaccgcctcagggggaggtggatgctgactgcatggatgtcaatgttcgaggaccagatggcttcacacccctcatgattgcctcctgcagtggagggggccttgagacaggcaacagtgaagaagaagaagatgcacctgctgtcatctctgacttcatctaccagggcgccagcttgcacaaccagacagaccgcaccggggagaccgccttgcacttggctgcccgatactctcgttcagatgctgcaaagcgcttgctggaggccagtgcagatgccaacatccaggacaacatgggccgtactccgttacatgcagcagtttctgcagatgctcagggtgtcttccagatcctgctccggaacagggccacagatctggatgcccgaatgcatgatggcacaactccactgatcctggctgcgcgcctggccgtggagggcatgctggaggacctcatcaactcacatgctgacgtcaatgccgtggatgacctaggcaagtcggctttgcattgggcggccgcggtgaacaatgtggatgctgctgttgtgctcctgaagaacggagccaacaaggacatgcagaacaacaaggaggagactcccctgttcctggccgcccgtgagggcagctatgagactgccaaagtgttgctggaccactttgccaaccgggacatcacggatcacatggaccgattgccgcgggacatcgcacaggagcgtatgcaccacgatatcgtgcggcttttggatgagtacaacctggtgcgcagcccacagctgcatggcactgccctgggtggcacacccactctgtctcccacactctgctcgcccaatggctacctgggcaatctcaagtctgccacacagggcaagaaggcccgcaagcccagcaccaaagggctggcttgtggtagcaaggaagctaaggacctcaaggcacggaggaagaagtcccaggatggcaagggctgcctgttggacagctcgacggatccggtaccagattacaaggacgacgatgacaagtaggaattcgaatttaaatcggatccgcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagacgccaccatggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatcccgcgctcagtcgtccaattctgccgtggacggcaccgccggacccggctccaccggatctcgctaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 7 - DNA, 5888 bp, Artificial Sequence
  plasmid construct
ggtaccgagctcttacgcgtgctagcccgggctcgagggccagattctacatgaatgttggtacgtatttatgtaaatgtatttttaaaacaaaagccaattgatatcttatgctttaatacttattccactaatcatttacatagatgcatcacgtgcagtaatcatttttattacctattcacaagcaaaaatattagtataaactgggtacaataaaatagagaaaagaaatattcaaatagataagcgttttgttaaaaaaaaaaaaaagaagaaagaaaggacacatttatcaggattcctatttcccgtacataatatggatgtttgtttgtttttgtaagttaacgggaccggtggtttaacttgttattgaaacatgctcgaaaaaatcaggtagcttattttgtaattgcttgttatgaaaccactggcatttctctggggaaaataagttaaaaactctttagctatcaggcagtgggttttaattttttatattggttaaatgtaacagtggatttgcgtactctctcctaatttctaactttgtgtaatcattcttgaaaccccaaatctagattttaaaaaagaagccttctaaaagttttcctgaagtttacttttcagttacaaagagtaaaataactttctgaaatgccttctgtaaatcgtggtggtggtgcggtttgtttggggagatttgttttgtttttaaaagacagtgcactttcttatgaaagagacagggaaagttttacctgtctgtctcctgggtttgttttttttttctttctttctttttcttttaaagattggtgataaggaattctaactacttaatgagatgggagaggcctcactccattggagtggaggagtccaggtggaagttgatggattggacaggtaaagagaagagtcccgcctcctcatgcctatagttgggtatatattaggaaaccttaaattatgtacagagagagaaagagagagagggacttgagttctgttatcttcttaagtagattcatattgtaagggtctcggggtgggggggttggcaaaatcctggagccagaagaaaggacagcagcattgatcaatcttacagctaacaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 8 - DNA, 4837 bp, Artificial Sequence
  plasmid construct
ggtaccgcaagtaaccacatgaacaaaaactcggggcaagtaaccacatgaacaagatctgcgatctaagtaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 9 - DNA, 4837 bp, Artificial Sequence
  plasmid construct
ggtaccgtaattaaccatattaacaaaaactcggggtaattaaccatattaacaagatctgcgatctaagtaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 10 - DNA, 21 bp, Artificial Sequence
  scramble shRNA
cctaaggttaagtcgccctcg

SEQ ID NO: 11 - DNA, 21 bp, Artificial Sequence
  shRNA target sequence
cctcagcagaactcactaaat

SEQ ID NO: 12 - DNA, 21 bp, Artificial Sequence
  shRNA target sequence
agttgcacttattgaccattt

SEQ ID NO: 13 - DNA, 21 bp, Artificial Sequence
  shRNA target sequence
tgaggagacctatgaggtatt

SEQ ID NO: 14 - DNA, 18 bp, Artificial Sequence
  primer
aggaagagacaggaaggc

SEQ ID NO: 15 - DNA, 17 bp, Artificial Sequence
  primer
tgtgtgctgaggaaggt

SEQ ID NO: 16 - DNA, 20 bp, Artificial Sequence
  primer
gctgcgaccagtacaagttg

SEQ ID NO: 17 - DNA, 19 bp, Artificial Sequence
  primer
ctgcctcgacgaactcctc

SEQ ID NO: 18 - DNA, 19 bp, Artificial Sequence
  primer
cctaatactggttcccact

SEQ ID NO: 19 - DNA, 19 bp, Artificial Sequence
  primer
cgccatcctctgtaacttc

SEQ ID NO: 20 - DNA, 19 bp, Artificial Sequence
  primer
gacagagtatgctcgctat

SEQ ID NO: 21 - DNA, 18 bp, Artificial Sequence
  primer
ctggctgacacctcaaag

SEQ ID NO: 22 - DNA, 20 bp, Artificial Sequence
  primer
gtctccaagcagctgaagcc

SEQ ID NO: 23 - DNA, 20 bp, Artificial Sequence
  primer
cctccatcaccgactggatc

SEQ ID NO: 24 - DNA, 22 bp, Artificial Sequence
  primer
aactccatcatgaagtgtgacg

SEQ ID NO: 25 - DNA, 20 bp, Artificial Sequence
  primer
gatccacatctgctggaagg

SEQ ID NO: 26 - DNA, 17 bp, Artificial Sequence
  primer
ttctgtgagcgagaccc

SEQ ID NO: 27 - DNA, 20 bp, Artificial Sequence
  primer
ggagattccatcagtcaccc

SEQ ID NO: 28 - DNA, 20 bp, Artificial Sequence
  primer
ctgagtctctgaggtcactt

SEQ ID NO: 29 - DNA, 17 bp, Artificial Sequence
  primer
tgataggatgctggggt

SEQ ID NO: 30 - DNA, 22 bp, Artificial Sequence
  primer
gaaggcccttcataaccaatga

SEQ ID NO: 31 - DNA, 25 bp, Artificial Sequence
  primer
gatatttggctaaggaggtgatgtc

SEQ ID NO: 32 - DNA, 22 bp, Artificial Sequence
  primer
tcagaggtggaagtggtaccct

SEQ ID NO: 33 - DNA, 16 bp, Artificial Sequence
  primer
gcagaggccggcattc

SEQ ID NO: 34 - DNA, 17 bp, Artificial Sequence
  primer
ttctgtgagcgagaccc

SEQ ID NO: 35 - DNA, 18 bp, Artificial Sequence
  primer
catcacacagcacatagc

SEQ ID NO: 36 - DNA, 17 bp, Artificial Sequence
  priemr
gtcagcaccaaacagcg

SEQ ID NO: 37 - DNA, 20 bp, Artificial Sequence
  primer
ggagattccatcagtcaccc

SEQ ID NO: 38 - DNA, 18 bp, Artificial Sequence
  primer
cgcacagactgacagaat

SEQ ID NO: 39 - DNA, 20 bp, Artificial Sequence
  primer
aggtaatccttgccatcgta

SEQ ID NO: 40 - DNA, 23 bp, Artificial Sequence
  primer
cctgttcttgatgattctctacc

SEQ ID NO: 41 - DNA, 18 bp, Artificial Sequence
  primer
ttgactgtgaggctaacg

SEQ ID NO: 42 - DNA, 18 bp, Artificial Sequence
  primer
acaactgggtgtcctttc

SEQ ID NO: 43 - DNA, 18 bp, Artificial Sequence
  primer
tctgctcaaactcctccg

SEQ ID NO: 44 - DNA, 17 bp, Artificial Sequence
  primer
aaagatagccgcttccc

SEQ ID NO: 45 - DNA, 27 bp, Artificial Sequence
  primer
gccgttcacaatcaatcaaatagttta

SEQ ID NO: 46 - DNA, 17 bp, Artificial Sequence
  primer
gctggaagatggacgca

SEQ ID NO: 47 - DNA, 20 bp, Artificial Sequence
  primer
attctcaatggtgtcactcg

SEQ ID NO: 48 - DNA, 20 bp, Artificial Sequence
  guide RNA for CRISPR/Cas9
caggaaggcgtccaattgcc